(19) United States
(12) Patent Application Publication
(10) Pub. No.: US 2026/0315437 A1
(43) Pub. Date: Oct. 15, 2026

(54) COMBINATION OF NAB-SIROLIMUS AND AN ESTROGEN SUPPRESOR FOR USE IN THE TREATMENT OF HORMONE-DEPENDENT CANCERS

(71) Applicant: AADI Bioscience, Inc., Morristown, NJ (US)

(72) Inventors: Shihe HOU, Millington, NJ (US); Andrew KWON, Manhattan Beach, CA (US); Brian James MCMORRAN, Woodland Hills, CA (US); Igor VIVANCO, London (GB)

(21) Appl. No.: 19/467,616

(22) Filed: Feb. 2, 2026

Related U.S. Application Data
(63) Continuation of application No. PCT/US2024/041303, filed on Aug. 7, 2024.
(60) Provisional application No. 63/531,511, filed on Aug. 8, 2023.

(51) Int. Cl. A61K 31/4196; A61K 31/436; A61P 35/00; A61P 35/04

(52) U.S. Cl. CPC A61K 31/4196; A61K 31/436; A61P 35/00; A61P 35/04

(57) ABSTRACT

The present application, in certain aspects, pertains to methods and compositions for the treatment of a hormone-dependent cancer (such as an endometrial cancer (e.g., endometrioid endometrial cancer) or hormone-receptor positive breast cancer), using a composition comprising nanoparticles comprising sirolimus and an albumin in combination with an estrogen suppressor (such as letrozole or fulvestrant).

### CROSS-REFERENCE TO RELATED APPLICATIONS

This application is a continuation of International Application No. PCT/US2024/041303, filed on Aug. 7, 2024, which claims the priority benefit of U.S. Provisional Application No. 63/531,511, filed on Aug. 8, 2023, the entire contents of each of which are hereby incorporated herein by reference for all purposes.

### FIELD OF THE INVENTION

The present application, in certain aspects, pertains to methods and compositions for the treatment of a hormone-dependent cancer using a composition comprising nanoparticles comprising an mTOR inhibitor (such as sirolimus) and an albumin in combination with an estrogen suppressor (such as letrozole or fulvestrant).

### BACKGROUND OF THE INVENTION

Hormone-dependent cancers, such as endometrial cancers (e.g., endometrioid endometrial cancer) or hormone-receptor positive cancers (e.g., hormone-receptor positive breast cancers), are difficult to treat as standard-of-care endocrine therapies lead to treatment resistance in most patients. Thus, there exist significant challenges with long-term treatment of patients with such current standard-of-care treatments.

mTOR inhibitors have found wide applications in treating diverse pathological conditions such as solid tumors, hematological malignancies, organ transplantation, restenosis, and rheumatoid arthritis. One such example is sirolimus (INN/USAN), also known as rapamycin—an immunosuppressant drug used to prevent rejection in organ transplantation. Sirolimus-eluting stents were approved in the United States to treat coronary restenosis. Additionally, sirolimus has been demonstrated as an effective inhibitor of tumor growth in various cell lines and animal models. Other limus drugs, such as analogs of sirolimus, have been designed to improve the pharmacokinetic and pharmacodynamic properties of sirolimus. For example, Temsirolimus was approved in the United States and Europe for the treatment of renal cell carcinoma. Everolimus was approved in the United States for treatment of advanced breast cancer, pancreatic neuroendocrine tumors, advanced renal cell carcinoma, and subependymal giant cell astrocytoma (SEGA) associated with Tuberous Sclerosis. The mode of action of sirolimus is to bind the cytosolic protein FK-binding protein 12 (FKBP12), and the sirolimus-FKBP12 complex in turn inhibits the mTOR pathway by directly binding to the mTOR Complex 1 (mTORC1).

Albumin-based nanoparticle compositions have been developed as a drug delivery system for delivering substantially water insoluble drugs. See, for example, U.S. Pat. Nos. 5,916,596; 6,506,405; 6,749,868, and 6,537,579, 7,820,788, and 7,923,536. Abraxane®, an albumin stabilized nanoparticle formulation of paclitaxel, was approved in the United States in 2005 and subsequently in various other countries for treating metastatic breast cancer. It was recently approved for treating non-small cell lung cancer in the United States, and has also shown therapeutic efficacy in various clinical trials for treating difficult-to-treat cancers such as bladder cancer and melanoma. Albumin derived from human blood has been used for the manufacture of Abraxane® as well as various other albumin-based nanoparticle compositions. Albumin-based nanoparticle composition comprising sirolimus, e.g., nab-sirolimus or Fyarro®, are known, e.g., U.S. Pat. Nos. 8,911,786 and 11,497,737.

There remains a continuing need in the art for advanced treatments of certain cancers including the difficult-to-treat hormone-dependent cancers, such as endometrial cancers (e.g., endometrioid endometrial cancer) or hormone-receptor positive cancers (e.g., hormone-receptor positive breast cancers).

### BRIEF DESCRIPTION OF THE DRAWINGS

FIGS. 1A and 1B show histograms of viable cell numbers (FIG. 1A) and cell death (FIG. 1B) for MDA-MB-361 cells.

FIGS. 2A and 2B show histograms of viable cell numbers (FIG. 2A) and cell death (FIG. 2B) for MCF7 cells.

FIG. 3 shows Western blot analyses for various markers (including p4EBP1 and AKT) from MCF7 cells receiving treatments comprising fulvestrant, nab-sirolimus, and combinations of fulvestrant and nab-sirolimus.

### BRIEF SUMMARY OF THE INVENTION

The present application, in certain aspects, provided is a method of treating a hormone-dependent cancer in an individual in need thereof, the method comprising administering to the individual: (a) a composition comprising nanoparticles comprising sirolimus and an albumin; and (b) an estrogen suppressor.

In some embodiments, the estrogen suppressor is an agent that suppresses estrogen production. In some embodiments, the estrogen suppressor is an aromatase inhibitor. In some embodiments, the estrogen suppressor is selected from the group consisting of letrozole, anastrozole, formestane, and exemestane. In some embodiments, the agent that suppresses estrogen production is letrozole. In some embodiments, the estrogen suppressor is an agent that suppresses estrogen activity. In some embodiments, the estrogen suppressor is an estrogen receptor antagonist. In some embodiments, the agent that suppresses estrogen activity is selected from the group consisting of fulvestrant, elacestrant, tamoxifen, hydroxyprogesterone caprate, droloxifene, ormeloxifene, toremifene, faloxifene, raloxifene, and clomiphene. In some embodiments, the agent that suppresses estrogen activity is fulvestrant. In some embodiments, the estrogen suppressor is administered to the individual orally, intramuscularly, intravenously, intraarterially, intraperitoneally, intravesicularly, subcutaneously, intrathecally, intrapulmonarily, intratracheally, intraocularly, transdermally, or by inhalation.

In some embodiments, the estrogen suppressor is administered to the individual orally. In some embodiments, the estrogen suppressor is administered to the individual intramuscularly.

In some embodiments, the estrogen suppressor is letrozole, and wherein letrozole is administered to the individual orally. In some embodiments, letrozole is administered to the individual at an amount of about 0.1 mg to about 10 mg. In some embodiments, letrozole is administered to the individual daily.

In some embodiments, the estrogen suppressor is fulvestrant, and wherein fulvestrant is administered to the individual intramuscularly. In some embodiments, fulvestrant is administered to the individual at an amount of about 100 mg to about 700 mg. In some embodiments, fulvestrant is administered to the individual on days 1, 15, and 29 of an initiation cycle followed by monthly maintenance cycles.

In some embodiments, the hormone-dependent cancer is an endometrioid endometrial cancer. In some embodiments, the hormone-dependent cancer is a breast cancer. In some embodiments, the breast cancer is a hormone receptor positive breast cancer. In some embodiments, the hormone-dependent cancer is locally advanced, advanced, malignant, advanced malignant, or metastatic. In some embodiments, the hormone-dependent cancer is refractory, relapsed, recurrent, or resistant to a prior treatment. In some embodiments, the prior treatment comprises an mTOR inhibitor and/or a composition comprising nanoparticles comprising an mTOR inhibitor and an albumin. In some embodiments, the prior treatment comprises a platinum agent and/or a checkpoint inhibitor. In some embodiments, the individual has not been treated with an mTOR inhibitor and/or a composition comprising nanoparticles comprising an mTOR inhibitor and an albumin. In some embodiments, the hormone-dependent cancer is stage III or stage IV.

In some embodiments, the individual is human.

In other aspects, provided herein is a method of treating an endometrial cancer in an individual in need thereof, the method comprising administering to the individual: (a) a composition comprising nanoparticles comprising sirolimus and an albumin; and (b) an estrogen suppressor, wherein the estrogen suppressor is an aromatase inhibitor, and wherein the estrogen suppressor is administered to the individual at an amount of about 0.1 mg to about 10 mg. In some embodiments, the endometrial cancer is an endometrioid endometrial cancer. In some embodiments, the estrogen suppressor is letrozole. In some embodiments, the estrogen suppressor is administered to the individual daily.

In other aspects, provided herein is a method of treating a hormone receptor positive breast cancer in an individual in need thereof, the method comprising administering to the individual: (a) a composition comprising nanoparticles comprising sirolimus and an albumin; and (b) an estrogen suppressor, wherein the estrogen suppressor is a selective estrogen receptor degrader (SERD), and wherein the estrogen suppressor is administered to the individual at an amount of about 200 mg to about 600 mg. In some embodiments, the estrogen suppressor is fulvestrant. In some embodiments, the estrogen suppressor is administered to the individual on days 1, 15, and 29 of an initiation cycle followed by monthly maintenance doses.

In some embodiments, sirolimus in the sirolimus nanoparticle composition is administered at an amount of about 10 mg/m² to about 150 mg/m². In some embodiments, sirolimus in the sirolimus nanoparticle composition is administered at an amount of about 100 mg/m². In some embodiments, sirolimus in the sirolimus nanoparticle composition is administered at an amount of about 75 mg/m². In some embodiments, sirolimus in the sirolimus nanoparticle composition is administered at an amount of about 56 mg/m². In some embodiments, sirolimus in the sirolimus nanoparticle composition is administered at an amount of about 45 mg/m². In some embodiments, sirolimus in the sirolimus nanoparticle composition is administered at an amount of about 30 mg/m².

In some embodiments, the sirolimus nanoparticle composition is administered twice out of every 3 weeks. In some embodiments, the sirolimus nanoparticle composition is administered on days 1 and 8 of a 21-day cycle. In some embodiments, the average diameter of the nanoparticles in the composition is no greater than about 150 nm. In some embodiments, the average diameter of the nanoparticles in the composition is no greater than about 120 nm. In some embodiments, the weight ratio of the albumin to sirolimus in the nanoparticle composition is no greater than about 9:1. In some embodiments, the nanoparticles comprise sirolimus associated with the albumin. In some embodiments, the nanoparticles comprise sirolimus coated with the albumin.

In some embodiments, the sirolimus nanoparticle composition is administered intravenously.

In some embodiments, the sirolimus nanoparticle composition is administered concurrently with the estrogen suppressor. In some embodiments, the sirolimus nanoparticle composition is administered sequentially with the estrogen suppressor. In some embodiments, the sirolimus nanoparticle composition is administered simultaneously with the estrogen suppressor.

The disclosures of all publications, patents, patent applications and published patent applications referred to herein are hereby incorporated herein by reference in their entirety.

### DETAILED DESCRIPTION OF THE INVENTION

The present application provides, in certain aspects, treatments for a hormone-dependent cancer (such as an endometrial cancer (e.g., endometrioid endometrial cancer) or hormone-receptor positive cancers (e.g., hormone-receptor positive breast cancers)) in an individual in need thereof, the methods comprising administering to the individual: (a) a composition comprising nanoparticles comprising an mTOR inhibitor (e.g., sirolimus) and an albumin; and (b) an estrogen suppressor. In some embodiments, the estrogen suppressor is an aromatase inhibitor, e.g., letrozole. In some embodiments, the estrogen suppressor is a selective estrogen receptor downregulator/degrader (SERD), e.g., fulvestrant.

The subject matter of the present application is based, at least in part, on the inventors' unique perspectives and findings that a combination of nab-sirolimus and an estrogen suppressor, e.g., an agent that suppresses estrogen (e.g., letrozole) or estrogen receptor signaling (e.g., fulvestrant), is useful for treating hormone-dependent cancer and overcoming commonly observed drug-resistance to standard-of-care endocrine therapies. For example, as reported herein, single-agent treatments with fulvestrant increased phosphorylated 4E-binding protein 1 (p4EBP1), a protein species known to be a driving force in tumorigenesis and also linked with very poor prognosis and drug resistance. Unexpectedly, the inventors demonstrated that treatment with nab-sirolimus and an estrogen suppressor, namely, fulvestrant, resulted in a decrease of p4EBP1. This finding is significant as it suggests that the combinations treatments taught herein are less prone to issues with drug resistance. Thus, these findings demonstrate that the combination treatments described herein solve a significant issue faced by currently available treatments.

Thus, provided herein, in certain aspects, is a method of treating a hormone-dependent cancer in an individual in need thereof, the method comprising administering to the individual: (a) a composition comprising nanoparticles comprising sirolimus and an albumin; and (b) an estrogen suppressor (such as an agent that suppresses estrogen, e.g., by suppressing estrogen production, or an agent that suppresses estrogen receptor activity, e.g., an antagonist). In other aspects, provided herein is a method of treating an endometrial cancer in an individual in need thereof, the method comprising administering to the individual: (a) a composition comprising nanoparticles comprising sirolimus and an albumin; and (b) an estrogen suppressor, wherein the estrogen suppressor is an aromatase inhibitor, and wherein the estrogen suppressor is administered to the individual at an amount of about 0.1 mg to about 10 mg. In some embodiments, the endometrial cancer is an endometrioid endometrial cancer. In some embodiments, the estrogen suppressor is letrozole. In some embodiments, the estrogen suppressor is administered to the individual daily.

In other aspects, provided herein is a method of treating an endometrial cancer in an individual in need thereof, the method comprising administering to the individual: (a) a composition comprising nanoparticles comprising sirolimus and an albumin; and (b) an estrogen suppressor, wherein the estrogen suppressor is an aromatase inhibitor, and wherein the estrogen suppressor is administered to the individual at an amount of about 0.1 mg to about 10 mg. In some embodiments, the endometrial cancer is an endometrioid endometrial cancer. In some embodiments, the estrogen suppressor is letrozole. In some embodiments, the estrogen suppressor is administered to the individual daily.

In other aspects, provided herein is a method of treating a hormone receptor positive breast cancer in an individual in need thereof, the method comprising administering to the individual: (a) a composition comprising nanoparticles comprising sirolimus and an albumin; and (b) an estrogen suppressor, wherein the estrogen suppressor is a selective estrogen receptor degrader (SERD), and wherein the estrogen suppressor is administered to the individual at an amount of about 200 mg to about 600 mg. In some embodiments, the estrogen suppressor is fulvestrant. In some embodiments, the estrogen suppressor is administered to the individual on days 1, 15, and 29 of an initiation cycle followed by monthly maintenance doses.

### I. Definitions

As used herein “nab” stands for nanoparticle albumin-bound, and “nab-sirolimus” is an albumin stabilized nanoparticle formulation of sirolimus. nab-sirolimus is also known as nab-rapamycin, which has been previously described. See, for example, U.S. Pat. Nos. 8,911,786 and 11,497,737, each of which is incorporated herein by reference in their entirety.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. In some embodiments, the treatment reduces the severity of one or more symptoms associated with cancer by at least about any of 10%, 20%, 30%, 40%, 50%, 60%, 70%, 80%, 90%, 95% or 100% compared to the corresponding symptom in the same subject prior to treatment or compared to the corresponding symptom in other subjects not receiving the treatment. Also encompassed by “treatment” is a reduction of pathological consequence of cancer. The methods of the invention contemplate any one or more of these aspects of treatment.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of subjects. Cancer development can be detectable using standard methods, including, but not limited to, computerized axial tomography (CAT scan), Magnetic Resonance Imaging (MRI), ultrasound, clotting tests, arteriography, biopsy, urine cytology, and cystoscopy. Development may also refer to cancer progression that may be initially undetectable and includes occurrence, recurrence, and onset.

The term “effective amount” used herein refers to an amount of a compound or composition sufficient to treat a specified disorder, condition or disease such as ameliorate, palliate, lessen, and/or delay one or more of its symptoms. In reference to cancer, an effective amount comprises an amount sufficient to cause a tumor to shrink and/or to decrease the growth rate of the tumor (such as to suppress tumor growth) or to prevent or delay other unwanted cell proliferation in cancer. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer.

As is understood in the art, an “effective amount” or “amount” may be in one or more doses, i.e., a single dose or multiple doses may be required to achieve the desired treatment endpoint. An effective amount may be considered in the context of administering one or more therapeutic agents, and a nanoparticle composition (e.g., a composition including sirolimus and an albumin) may be considered to be given in an effective amount if, in conjunction with one or more other agents, a desirable or beneficial result may be or is achieved. The components (e.g., the first and second therapies) in a combination therapy of the invention may be administered sequentially, simultaneously, or concurrently using the same or different routes of administration for each component. Thus, an effective amount of a combination therapy includes an amount of the first therapy and an amount of the second therapy that when administered sequentially, simultaneously, or concurrently produces a desired outcome.

“In conjunction with” or “in combination with” refers to administration of one treatment modality in addition to another treatment modality, such as administration of a nanoparticle composition described herein in addition to administration of the other agent to the same individual under the same treatment plan. As such, “in conjunction with” or “in combination with” refers to administration of one treatment modality before, during or after delivery of the other treatment modality to the individual.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy is contained in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

As used herein, the term “individual” refers to a mammal and includes, but is not limited to, human, bovine, horse, feline, canine, rodent, rat, mouse, dog, or primate. In some embodiments, the individual is a human individual.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Those skilled in the art will recognize that several embodiments are possible within the scope and spirit of the present disclosure. The following description illustrates the disclosure and, of course, should not be construed in any way as limiting the scope of the inventions described herein.

### II. Methods of Treatment

Provided herein are methods for the treatment of a hormone-dependent cancer in an individual in need thereof, the method comprising administering to the individual: (a) a composition comprising nanoparticles comprising sirolimus and an albumin; and (b) an estrogen suppressor, such as an agent that suppresses estrogen or estrogen receptor signaling.

In some embodiments, provided is a method of treating a hormone-dependent cancer, such as an endometrial cancer (e.g., endometrioid endometrial cancer), in an individual in need thereof, the method comprising administering to the individual: (a) a composition comprising nanoparticles comprising sirolimus and an albumin; and (b) an estrogen suppressor, wherein the estrogen suppressor is an aromatase inhibitor, e.g., letrozole. In some embodiments, the aromatase inhibitor is letrozole. In some embodiments, the aromatase inhibitor is administered to the individual at an amount of about 0.1 mg to about 10 mg, e.g., at about 2.5 mg. In some embodiments, the aromatase inhibitor is administered orally (i.e., the aromatase inhibitor, e.g., letrozole, is suitable for oral administration). In some embodiments, the aromatase inhibitor is administered daily. In some embodiments, the composition comprising nanoparticles comprising sirolimus and the albumin, e.g., nab-sirolimus, is administered to the individual at an amount of about 70 mg/m² to about 110 mg/m², such as about 100 mg/m². In some embodiments, the composition comprising nanoparticles comprising sirolimus and the albumin, e.g., nab-sirolimus, is administered intravenously. In some embodiments, the composition comprising nanoparticles comprising sirolimus and the albumin, e.g., nab-sirolimus, is administered to the individual weekly, such as two out of every three weeks, e.g., on days 1 and 8 of a 21-day cycle. In some embodiments, the individual is human.

In some embodiments, provided is a method of treating a hormone-dependent cancer, such as a hormone receptor positive breast cancer, in an individual in need thereof, the method comprising administering to the individual: (a) a composition comprising nanoparticles comprising sirolimus and an albumin; and (b) an estrogen suppressor, wherein the estrogen suppressor is a selective estrogen receptor degrader (SERD), e.g., fulvestrant. In some embodiments, the SERD is administered to the individual at an amount of about 200 mg to about 600 mg, e.g., 500 mg. In some embodiments, the SERD is administered intramuscularly (i.e., SERD, e.g., fulvestrant, is suitable for intramuscular administration). In some embodiments, the SERD is administered to the individual on days 1, 15, and 29 of an initiation cycle followed by monthly maintenance doses. In some embodiments, the composition comprising nanoparticles comprising sirolimus and the albumin, e.g., nab-sirolimus, is administered to the individual at an amount of about 70 mg/m² to about 110 mg/m², such as about 100 mg/m². In some embodiments, the composition comprising nanoparticles comprising sirolimus and the albumin, e.g., nab-sirolimus, is administered intravenously. In some embodiments, the composition comprising nanoparticles comprising sirolimus and the albumin, e.g., nab-sirolimus, is administered to the individual weekly, such as two out of every three weeks, e.g., on days 1 and 8 of a 21-day cycle. In some embodiments, the individual is human.

Further discussion of the methods, and aspects thereof, taught herein is included in the sections below. The modular discussion of such components does not limit the scope of the invention and one of ordinary skill in the art will readily appreciate how certain features from the sections below can be combined to form the combination treatments and associated subject matter taught herein.

### A. Hormone-Dependent Cancers

In some embodiments, the hormone-dependent cancer is a hormone-receptor positive cancer, such as a cancer that is estrogen receptor positive and/or progesterone receptor positive. In some embodiments, the hormone-dependent cancer is hormone dependent (e.g., relies on hormone-driven development and/or growth), such as estrogen dependent. Techniques for determining hormone dependency and hormone receptor presence are known in the art, e.g., obtaining a biopsy to test for, e.g., the presence of estrogen or progesterone receptor using techniques such as immunohistochemistry (IHC). In some embodiments, the hormone-dependent cancer is an endometrial cancer, such as endometrioid endometrial cancer (EEC), e.g., an estrogen-dependent endometrial cancer including an estrogen-dependent endometrioid endometrial cancer. In some embodiments, the endometrial cancer (e.g., endometrioid endometrial cancer) is a hormone receptor positive endometrial cancer (e.g., endometrioid endometrial cancer), such as an endometrioid endometrial cancer positive for estrogen receptors and/or progesterone receptors. In some embodiments, the hormone-dependent cancer is a breast cancer. In some embodiments, the breast cancer is a hormone receptor positive breast cancer, such as a breast cancer positive for estrogen receptors and/or progesterone receptors.

In some embodiments, the hormone-dependent cancer is locally advanced, advanced, malignant, advanced malignant, or metastatic. In some embodiments, the advanced hormone-dependent cancer is unresectable or not resectable without risk of serious injury or a heightened risk of death. In some embodiments, the hormone-dependent cancer is refractory, relapsed, recurrent, or resistant to a prior treatment. In some embodiments, the prior treatment comprises an mTOR inhibitor (such as sirolimus) and/or a composition comprising nanoparticles comprising an mTOR inhibitor (such as sirolimus) and an albumin. In some embodiments, the prior treatment comprises a chemotherapy, immunotherapy, targeted therapy, and/or checkpoint inhibitor. In some embodiments, the chemotherapy comprises a platinum agent. In some embodiments, the prior treatment comprises a platinum agent and/or a checkpoint inhibitor. In some embodiments, the individual has received zero or 1 or more prior treatments, such as 0-1 or more chemotherapy regimens. In some embodiments, the individual has cancer that is recurrent, locally advanced, or metastatic. In some embodiments, the individual has received 1 or more lines of chemotherapy. In some embodiments, the individual has received 0 line of chemotherapy, or is chemotherapy-naïve. In some embodiments, the prior therapy is an adjuvant therapy. In some embodiments, the prior adjuvant therapy is a chemotherapy, a hormonal therapy, or a checkpoint inhibitor. In some embodiments, the adjuvant therapy is completed ≥6 months prior to treatment according to the method described herein. In some embodiments, the prior therapy is a non-chemotherapy-based treatment. In some embodiments, the prior non-chemotherapy-based ended ≥4 months prior to treatment according to the method described herein.

In some embodiments, the individual has not been treated with an mTOR inhibitor (such as sirolimus) and/or a composition comprising nanoparticles comprising an mTOR inhibitor (such as sirolimus) and an albumin.

In some embodiments, the hormone-dependent cancer is stage III or stage IV, such as according to the International Federation of Gynecology and Obstetrics (FIGO) staging.

In some embodiments, the hormone-dependent cancer is advanced or recurrent endometrial cancer (e.g., endometrioid endometrial cancer). In some embodiments, the advanced recurrent endometrial cancer (e.g., endometrioid endometrial cancer) is unresectable FIGO stage III or IV. In some embodiments, the recurrent endometrial cancer (e.g., endometrioid endometrial cancer) is grade 3 or higher, wherein the individual was previously treated with a platinum agent and/or a checkpoint inhibitor.

In some embodiments, the hormone-dependent cancer is an ovarian cancer, such as an ovarian cancer being estrogen receptor positive and/or progesterone receptor positive. In some embodiments, the hormone-dependent cancer is a cervical cancer, such as a cervical cancer being estrogen receptor positive and/or progesterone receptor positive.

### B. Dosing and Methods of Administration

The dose of the mTOR inhibitor nanoparticle composition (such as sirolimus/albumin nanoparticle composition) administered to an individual (e.g., a human) may vary with the particular composition, the method of administration, the hormone-dependent cancer being treated, and the particular stage of tumor being treated. The amount should be sufficient to produce a desirable response, such as a therapeutic or prophylactic response against the tumor. In some embodiments, the amount of mTOR inhibitor (such as a limus drug, e.g., sirolimus or a derivative thereof) in the composition is below the level that induces a toxicological effect (e.g., an effect above a clinically acceptable level of toxicity) or is at a level where a potential side effect can be controlled or tolerated when the mTOR inhibitor nanoparticle composition is administered to the individual.

In some embodiments, when the mTOR inhibitor nanoparticle composition (such as sirolimus/albumin nanoparticle composition) is administered, the mTOR inhibitor nanoparticle composition is administered to the individual simultaneously with an estrogen suppressor (e.g., letrozole or fulvestrant) for at least one administration (e.g., due cycle differences of different agents it may be certain administration can be performed simultaneously while other cannot be). For example, the mTOR inhibitor nanoparticle compositions and an estrogen suppressor (e.g., letrozole or fulvestrant) are administered with a time separation of no more than about 15 minutes, such as no more than about any of 10, 5, or 1 minutes. In one example, wherein the compounds are in solution, simultaneous administration can be achieved by administering a solution containing the combination of compounds. In another example, simultaneous administration of separate solutions or compositions, one of which contains the mTOR inhibitor nanoparticle composition (such as sirolimus/albumin nanoparticle composition) and the other of which contains an estrogen suppressor (e.g., letrozole or fulvestrant), can be employed. In one example, simultaneous administration can be achieved by administering a composition containing the combination of compounds. In another example, simultaneous administration can be achieved by administering two separate compositions, one comprising the mTOR inhibitor nanoparticle composition (such as sirolimus/albumin nanoparticle composition administered intravenously) and the other comprising an estrogen suppressor (e.g., letrozole or fulvestrant). In some embodiments, simultaneous administration of the mTOR inhibitor (such as a limus drug, e.g., sirolimus or a derivative thereof) in the nanoparticle composition and an estrogen suppressor (e.g., letrozole or fulvestrant) can be combined with supplemental doses of the mTOR inhibitor and/or the estrogen suppressor (e.g., letrozole or fulvestrant).

In other embodiments, the mTOR inhibitor nanoparticle composition (such as sirolimus/albumin nanoparticle composition) and an estrogen suppressor (e.g., letrozole or fulvestrant) are not administered simultaneously for at least one administration. In some embodiments, the mTOR inhibitor nanoparticle composition (such as sirolimus/albumin nanoparticle composition) is administered before an estrogen suppressor (e.g., letrozole or fulvestrant). In other embodiments, the estrogen suppressor (e.g., letrozole or fulvestrant) is administered before the mTOR inhibitor nanoparticle composition (such as sirolimus/albumin nanoparticle composition). The time difference in non-simultaneous administrations can be greater than 1 minute, five minutes, 10 minutes, 15 minutes, 30 minutes, 45 minutes, 60 minutes, two hours, three hours, six hours, nine hours, 12 hours, 24 hours, 36 hours, or 48 hours. In other embodiments, the first administered compound is provided time to take effect on the patient before the second administered compound is administered. In some embodiments, the difference in time does not extend beyond the time for the first administered compound to complete its effect in the patient, or beyond the time the first administered compound is completely or substantially eliminated or deactivated in the patient.

In some embodiments, the administration of the mTOR inhibitor nanoparticle composition (such as sirolimus/albumin nanoparticle composition) and an estrogen suppressor (e.g., letrozole or fulvestrant) are concurrent for at least one administration, i.e., the administration period of the mTOR inhibitor nanoparticle composition and that of the estrogen suppressor (e.g., letrozole or fulvestrant) overlap with each other. In some embodiments, the mTOR inhibitor nanoparticle composition (such as sirolimus/albumin nanoparticle composition) is administered for at least one cycle (for example, at least any of 2, 3, 4, 5, 6, 7, 8, 9, 10, 11, 12, 13, 14 15, 16, 17, 18, 19, or 20 cycles) prior to the administration of an estrogen suppressor (e.g., letrozole or fulvestrant). In some embodiments, the estrogen suppressor (e.g., letrozole or fulvestrant) is administered for at least any of one, two, three, or four weeks. In some embodiments, the administration of the mTOR inhibitor nanoparticle composition (such as sirolimus/albumin nanoparticle composition) and an estrogen suppressor (e.g., letrozole or fulvestrant) are initiated at about the same time (for example, within any one of 1 day, 2 days, 3 days, 4 days, 5 days, 6 days, 1 week, 2 weeks, or 3 weeks). In some embodiments, the administration of the mTOR inhibitor nanoparticle composition (such as sirolimus/albumin nanoparticle composition) and an estrogen suppressor (e.g., letrozole or fulvestrant) are terminated at about the same time (for example, within any one of 1 day, 2 days, 3 days, 4 days, 5 days, 6 days, 1 week, 2 weeks, or 3 weeks). In some embodiments, the administration of an estrogen suppressor (e.g., letrozole or fulvestrant) continues (for example for about any one of 1, 2, 3, 4, 5, 6, 7, 8, 9, 10, 11, or 12 months) after the termination of the administration of the mTOR inhibitor nanoparticle composition (such as sirolimus/albumin nanoparticle composition). In some embodiments, the administration of an estrogen suppressor (e.g., letrozole or fulvestrant) is initiated after (for example after about any one of 1, 2, 3, 4, 5, 6, 7, 8, 9, 10, 11, or 12 months) the initiation of the administration of the mTOR inhibitor nanoparticle composition (such as sirolimus/albumin nanoparticle composition). In some embodiments, the administrations of the mTOR inhibitor nanoparticle composition (such as sirolimus/albumin nanoparticle composition) and an estrogen suppressor (e.g., letrozole or fulvestrant) are initiated and terminated at about the same time. In some embodiments, the administrations of the mTOR inhibitor nanoparticle composition (such as sirolimus/albumin nanoparticle composition) and an estrogen suppressor (e.g., letrozole or fulvestrant) are initiated at about the same time and the administration of the estrogen suppressor (e.g., letrozole or fulvestrant) continues (for example for about any one of 1, 2, 3, 4, 5, 6, 7, 8, 9, 10, 11, or 12 months) after the termination of the administration of the mTOR inhibitor nanoparticle composition. In some embodiments, the administration of the mTOR inhibitor nanoparticle composition (such as sirolimus/albumin nanoparticle composition) and an estrogen suppressor (e.g., letrozole or fulvestrant) stop at about the same time and the administration of the estrogen suppressor (e.g., letrozole or fulvestrant) is initiated after (for example after about any one of 1, 2, 3, 4, 5, 6, 7, 8, 9, 10, 11, or 12 months) the initiation of the administration of the mTOR inhibitor nanoparticle composition.

In some embodiments, the estrogen suppressor (e.g., letrozole or fulvestrant) is administered according to manufacture's instructions for a single-agent treatment or another combination treatment not comprising nab-sirolimus. In some embodiments, the administration of an estrogen suppressor (e.g., letrozole or fulvestrant) is reduced for a patient consideration, such as an adverse event. In some embodiments, the reduction in administration of an estrogen suppressor (e.g., letrozole or fulvestrant) is for only a portion of the treatment, e.g., until the individual recovers (at least partially) from the adverse event. In some embodiments, the estrogen suppressor (e.g., letrozole or fulvestrant) is administered at what is considered a sub-therapeutic dose for single-agent use of the estrogen suppressor (e.g., the combination treatments taught herein enable use of lower doses of one or both therapeutic agents to provide efficacious treatment).

In some embodiments, the administration of the composition comprising an mTOR inhibitor and an albumin and an estrogen suppressor (e.g., letrozole or fulvestrant) continues for at least any of 1, 2, 3, 4, 5, 6, 7, 8, 9, 10, 11, 12, 13, 14 15, 16, 17, 18, 19, or 20 cycles.

In some embodiments, the administration of the mTOR inhibitor nanoparticle composition (such as sirolimus/albumin nanoparticle composition) and an estrogen suppressor (e.g., letrozole or fulvestrant) are non-concurrent. For example, in some embodiments, the administration of the mTOR inhibitor nanoparticle composition (such as sirolimus/albumin nanoparticle composition) is terminated before an estrogen suppressor (e.g., letrozole or fulvestrant) is administered. In some embodiments, the administration of an estrogen suppressor (e.g., letrozole or fulvestrant) is terminated before the mTOR inhibitor nanoparticle composition (such as sirolimus/albumin nanoparticle composition) is administered. The time period between these two non-concurrent administrations can range from about two to eight weeks, such as about four weeks.

The dosing frequency of the mTOR inhibitor nanoparticle composition (such as sirolimus/albumin nanoparticle composition) and an estrogen suppressor (e.g., letrozole or fulvestrant) may be adjusted over the course of the treatment, based on the judgment of the administering physician. When administered separately, the mTOR inhibitor nanoparticle composition (such as sirolimus/albumin nanoparticle composition) and an estrogen suppressor (e.g., letrozole or fulvestrant) can be administered at different dosing frequency or intervals. For example, the mTOR inhibitor nanoparticle composition (such as sirolimus/albumin nanoparticle composition) can be administered once every three weeks, while the estrogen suppressor (e.g., letrozole or fulvestrant) can be administered more or less frequently, e.g., daily. In some embodiments, a sustained continuous release formulation of the nanoparticle and/or an estrogen suppressor (e.g., letrozole or fulvestrant) may be used. Various formulations and devices for achieving sustained release are known in the art. A combination of the administration configurations described herein can also be used.

The mTOR inhibitor nanoparticle composition (such as sirolimus/albumin nanoparticle composition) and an estrogen suppressor (e.g., letrozole or fulvestrant) can be administered using the same route of administration or different routes of administration. In some embodiments (for both simultaneous and sequential administrations), the mTOR inhibitor (such as a limus drug, e.g., sirolimus or a derivative thereof) in the mTOR inhibitor nanoparticle composition and an estrogen suppressor (e.g., letrozole or fulvestrant) are administered at a predetermined ratio.

The doses required for the mTOR inhibitor (such as a limus drug, e.g., sirolimus or a derivative thereof) in the mTOR inhibitor nanoparticle composition and/or an estrogen suppressor (e.g., letrozole or fulvestrant) may (but not necessarily) be the same or lower than what is normally required when each agent is administered alone. Thus, in some embodiments, a subtherapeutic amount of the mTOR inhibitor (such as a limus drug, e.g., sirolimus or a derivative thereof) in the mTOR inhibitor nanoparticle composition and/or an estrogen suppressor (e.g., letrozole or fulvestrant). “Subtherapeutic amount” or “subtherapeutic level” refer to an amount that is less than the therapeutic amount, that is, less than the amount normally used when the mTOR inhibitor nanoparticle composition (such as sirolimus/albumin nanoparticle composition) and/or an estrogen suppressor (e.g., letrozole or fulvestrant) are administered alone. The reduction may be reflected in terms of the amount administered at a given administration and/or the amount administered over a given period of time (reduced frequency). For example, in some embodiments, the method comprises administering a composition comprising nanoparticles comprising an mTOR inhibitor and an albumin at a dosage of less than about 100 mg/m², such as about any of 90 mg/m², 80 mg/m², 70 mg/m², 60 mg/m², 50 mg/m², 40 mg/m², 30 mg/m², 20 mg/m², or 10 mg/m².

In some embodiments, enough of an estrogen suppressor (e.g., letrozole or fulvestrant) is administered so as to allow reduction of the normal dose of the mTOR inhibitor (such as a limus drug, e.g., sirolimus or a derivative thereof) in the mTOR inhibitor nanoparticle composition required to affect the same degree of treatment by at least about any of 5%, 10%, 20%, 30%, 50%, 60%, 70%, 80%, 90%, or more. In some embodiments, enough of the mTOR inhibitor (such as a limus drug, e.g., sirolimus or a derivative thereof) in the mTOR inhibitor nanoparticle composition is administered so as to allow reduction of the normal dose of the an estrogen suppressor (e.g., letrozole or fulvestrant) required to affect the same degree of treatment by at least about any of 5%, 10%, 20%, 30%, 50%, 60%, 70%, 80%, 90%, or more.

In some embodiments, the dose of both the mTOR inhibitor (such as a limus drug, e.g., sirolimus or a derivative thereof) in the mTOR inhibitor nanoparticle composition and an estrogen suppressor (e.g., letrozole or fulvestrant) are reduced as compared to the corresponding normal dose of each when administered alone. In some embodiments, both the mTOR inhibitor (such as a limus drug, e.g., sirolimus or a derivative thereof) in the mTOR inhibitor nanoparticle composition and an estrogen suppressor (e.g., letrozole or fulvestrant) are administered at a subtherapeutic, i.e., reduced, level. In some embodiments, the dose of the mTOR inhibitor (such as a limus drug, e.g., sirolimus or a derivative thereof) in the mTOR inhibitor nanoparticle composition and/or an estrogen suppressor (e.g., letrozole or fulvestrant) is substantially less than the established maximum toxic dose (MTD). For example, the dose of the mTOR inhibitor nanoparticle composition (such as sirolimus/albumin nanoparticle composition) and/or an estrogen suppressor (e.g., letrozole or fulvestrant) is less than about 50%, 40%, 30%, 20%, or 10% of the MTD.

A combination of the administration configurations described herein can be used. The combination therapy methods described herein may be performed alone or in conjunction with another therapy, such as surgery, radiation, gene therapy, immunotherapy, bone marrow transplantation, stem cell transplantation, hormone therapy, targeted therapy, cryotherapy, ultrasound therapy, photodynamic therapy, and/or chemotherapy and the like. Additionally, a person having a greater risk of developing the hormone-dependent cancer may receive treatments to inhibit and/or delay the development of the disease.

As will be understood by those of ordinary skill in the art, in some embodiments, the appropriate doses of second agents will be approximately those already employed in clinical therapies wherein an agent that suppresses estrogen (e.g., letrozole or fulvestrant) is administered alone or in combination with other chemotherapeutic agents. Variation in dosage will likely occur depending on the condition being treated. As described above, in some embodiments, the second chemotherapeutic agent may be administered at a reduced level.

In some embodiments, the amounts of the mTOR inhibitor nanoparticle composition (such as sirolimus/albumin nanoparticle composition) and an estrogen suppressor (e.g., letrozole or fulvestrant) are below the levels that induce a toxicological effect (i.e., an effect above a clinically acceptable level of toxicity) or are at a level where a potential side effect can be controlled or tolerated when the mTOR inhibitor nanoparticle composition and an estrogen suppressor (e.g., letrozole or fulvestrant) are administered to the individual.

In some embodiments, the amount of the mTOR inhibitor nanoparticle composition (such as sirolimus/albumin nanoparticle composition) is close to a maximum tolerated dose (MTD) of the composition following the same dosing regimen when administered with an estrogen suppressor (e.g., letrozole or fulvestrant). In some embodiments, the amount of the mTOR inhibitor nanoparticle composition (such as sirolimus/albumin nanoparticle composition) is more than about any of 80%, 90%, 95%, or 98% of the MTD when administered with an estrogen suppressor (e.g., letrozole or fulvestrant).

As described herein, in some embodiments, reference to amounts of a composition comprising nanoparticles comprising an mTOR inhibitor and an albumin are based on the amount of the mTOR inhibitor therein. In some embodiments, the amount of an mTOR inhibitor (such as a limus drug, e.g., sirolimus) in the mTOR inhibitor nanoparticle composition is about any of 25 mg/m², 30 mg/m², 45 mg/m², 50 mg/m², 56 mg/m², 60 mg/m², 75 mg/m², 80 mg/m², 90 mg/m², 100 mg/m², 120 mg/m², 160 mg/m², 175 mg/m², 180 mg/m², 200 mg/m², 210 mg/m², 220 mg/m², 250 mg/m², 260 mg/m², 300 mg/m², 350 mg/m², 400 mg/m², 500 mg/m², 540 mg/m², 750 mg/m², 1000 mg/m², or 1080 mg/m² mTOR inhibitor. In some embodiments, the mTOR inhibitor nanoparticle composition includes less than about any of 350 mg/m², 300 mg/m², 250 mg/m², 200 mg/m², 150 mg/m², 120 mg/m², 100 mg/m², 90 mg/m², 50 mg/m², or 30 mg/m² mTOR inhibitor (such as a limus drug, e.g., sirolimus). In some embodiments, the amount of the mTOR inhibitor (such as a limus drug, e.g., sirolimus) per administration is less than about any of 40 mg/m², 39 mg/m², 38 mg/m², 37 mg/m², 36 mg/m², 35 mg/m², 34 mg/m², 33 mg/m², 32 mg/m², 31 mg/m², 30 mg/m², 29 mg/m², 28 mg/m², 27 mg/m², 26 mg/m², 25 mg/m², 24 mg/m², 23 mg/m², 22 mg/m², 21 mg/m², 20 mg/m², 19 mg/m², 18 mg/m², 17 mg/m², 16 mg/m², 15 mg/m², 14 mg/m², 13 mg/m², 12 mg/m², 11 mg/m², 10 mg/m², 9 mg/m², 8 mg/m², 7 mg/m², 6 mg/m², 5 mg/m², 4 mg/m², 3 mg/m², 2 mg/m², or 1 mg/m². In some embodiments, the mTOR inhibitor (such as a limus drug, e.g., sirolimus) in the mTOR inhibitor nanoparticle composition is included in any of the following ranges: about 1 to about 5 mg/m², about 5 to about 10 mg/m², about 10 to about 25 mg/m², about 25 to about 50 mg/m², about 50 to about 75 mg/m², about 75 to about 100 mg/m², about 100 to about 125 mg/m², about 125 to about 150 mg/m², about 150 to about 175 mg/m², about 175 to about 200 mg/m², about 200 to about 225 mg/m², about 225 to about 250 mg/m², about 250 to about 300 mg/m², about 300 to about 350 mg/m², or about 350 to about 400 mg/m². In some embodiments, the mTOR inhibitor (such as a limus drug, e.g., sirolimus) in the mTOR inhibitor nanoparticle composition is about 30 to about 300 mg/m², such as about 100 to about 150 mg/m², about 120 mg/m2, about 130 mg/m², or about 140 mg/m². In some embodiments, the amount of the mTOR inhibitor nanoparticle composition is administered every four weeks (e.g., day 1 of a 28-day cycle). In some embodiments, the amount of the mTOR inhibitor nanoparticle composition is administered every three weeks (e.g., day 1 of a 21-day cycle). In some embodiments, the amount of the mTOR inhibitor nanoparticle composition is administered every two weeks (e.g., day 1 of a 14-day cycle). In some embodiments, the amount of the mTOR inhibitor nanoparticle composition is administered weekly. In some embodiments, the amount of the mTOR inhibitor nanoparticle composition is administered weekly every 2 out of 3 weeks. In some embodiments, the amount of the mTOR inhibitor nanoparticle composition is on days 8 and 15 of a 21-day cycle, days 1 or 8 of a 21-day cycle, days 15 and 21 or a 21-day cycle, days 1 and 15 of a 21-day cycle, or days 1 and 21 of a 21-day cycle.

In some embodiments, the estrogen suppressor (e.g., letrozole or fulvestrant) is administered at about 0.1 mg to about 2,500 mg, including any of about 100 mg to about 900 mg, about 200 mg to about 800 mg, about 200 mg to about 700 mg, about 200 mg to about 600 mg, or about 200 mg to about 400 mg. In some embodiments, the estrogen suppressor (e.g., letrozole or fulvestrant) is administered at about 2,500 mg or less, such as about any of 2,250 mg or less, 2,000 mg or less, 1,750 mg or less, 1,500 mg or less, 1,250 mg or less, 1,000 mg or less, 750 mg or less, 700 mg or less, 650 mg or less, 600 mg or less, 550 mg or less, 500 mg or less, 450 mg or less, 400 mg or less, 350 mg or less, 300 mg or less, 250 mg or less, 200 mg or less, 150 mg or less, 100 mg or less, 90 mg or less, 80 mg or less, 70 mg or less, 60 mg or less, 50 mg or less, 40 mg or less, 30 mg or less, 20 mg or less, 10 mg or less, 9 mg or less, 8 mg or less, 7 mg or less, 6 mg or less, 5 mg or less, 4.5 mg or less, 4 mg or less, 3.5 mg or less, 3 mg or less, 2.5 mg or less, 2 mg or less, 1.5 mg or less, 1 mg or less, 0.9 mg or less, 0.8 mg or less, 0.7 mg or less, 0.6 mg or less, 0.5 mg or less, 0.4 mg or less, 0.3 mg or less, 0.2 mg or less, or 0.1 mg or less. In some embodiments, the estrogen suppressor (e.g., letrozole or fulvestrant) is administered at about any of 2,500 mg, 2,400 mg, 2,300 mg, 2,200 mg, 2,100 mg, 2,000 mg, 1,900 mg, 1,800 mg, 1,700 mg, 1,600 mg, 1,500 mg, 1,400 mg, 1,300 mg, 1,200 mg, 1,100 mg, 1,000 mg, 900 mg, 800 mg, 750 mg, 700 mg, 650 mg, 600 mg, 550 mg, 500 mg, 450 mg, 400 mg, 350 mg, 300 mg, 250 mg, 200 mg, 150 mg, 100 mg, 90 mg, 80 mg, 70 mg, 60 mg, 50 mg, 40 mg, 30 mg, 20 mg, 10 mg, 9 mg, 8 mg, 7 mg, 6 mg, 5 mg, 4.5 mg, 4 mg, 3.5 mg, 3 mg, 2.5 mg, 2 mg, 1.5 mg, 1 mg, 0.9 mg, 0.8 mg, 0.7 mg, 0.6 mg, 0.5 mg, 0.4 mg, 0.3 mg, 0.2 mg, or 0.1 mg.

In some embodiments, the dosing frequencies for the administration of the mTOR inhibitor nanoparticle composition (such as sirolimus/albumin nanoparticle composition) include, but are not limited to, daily, every two days, every three days, every four days, every five days, every six days, weekly without break, three out of four weeks (such as on days 1, 8, and 15 of a 28-day cycle), once every three weeks, once every two weeks, or two out of three weeks. In some embodiments, the mTOR inhibitor nanoparticle composition (such as sirolimus/albumin nanoparticle composition) is administered about once every 2 weeks, once every 3 weeks, once every 4 weeks, once every 6 weeks, or once every 8 weeks. In some embodiments, the mTOR inhibitor nanoparticle composition (such as sirolimus/albumin nanoparticle composition) is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, or eleven times. In some embodiments, the dosing frequency is once every two days for five times. In some embodiments, the mTOR inhibitor (such as a limus drug, e.g., sirolimus or a derivative thereof) is administered over a period of at least ten days, wherein the interval between each administration is no more than about two days, and wherein the dose of the mTOR inhibitor at each administration is about 0.25 mg/m² to about 250 mg/m², about 0.25 mg/m² to about 150 mg/m², about 0.25 mg/m² to about 75 mg/m², such as about 0.25 mg/m² to about 25 mg/m², or about 25 mg/m² to about 50 mg/m².

The administration of the mTOR inhibitor nanoparticle composition (such as sirolimus/albumin nanoparticle composition) can be extended over an extended period of time, such as from about a month up to about seven years. In some embodiments, the mTOR inhibitor nanoparticle composition (such as sirolimus/albumin nanoparticle composition) is administered over a period of at least about any of 2, 3, 4, 5, 6, 7, 8, 9, 10, 11, 12, 18, 24, 30, 36, 48, 60, 72, or 84 months.

In some embodiments, the dosage of an mTOR inhibitor (such as a limus drug, e.g., sirolimus or a derivative thereof) in a nanoparticle composition can be in the range of 5-400 mg/m² when given on a 3-week schedule, or 5-250 mg/m² (such as 80-150 mg/m², for example 100-120 mg/m²) when given on a weekly schedule. For example, the amount of an mTOR inhibitor (such as a limus drug, e.g., sirolimus or a derivative thereof) is about 60 to about 300 mg/m² (e.g., about 260 mg/m²) on a 3-week schedule.

In some embodiments, the exemplary dosing schedules for the administration of the mTOR inhibitor nanoparticle composition (such as sirolimus/albumin nanoparticle composition) include, but are not limited to, 100 mg/m², weekly, without break; 10 mg/m² weekly, 3 out of four weeks (such as on days 1, 8, and 15 of a 28-day cycle); 45 mg/m² weekly, 3 out of four weeks (such as on days 1, 8, and 15 of a 28-day cycle); 75 mg/m² weekly, 3 out of four weeks (such as on days 1, 8, and 15 of a 28-day cycle); 100 mg/m², weekly, 3 out of 4 weeks; 125 mg/m², weekly, 3 out of 4 weeks; 125 mg/m², weekly, 2 out of 3 weeks; 130 mg/m², weekly, without break; 175 mg/m², once every 2 weeks; 260 mg/m², once every 2 weeks; 260 mg/m², once every 3 weeks; 180-300 mg/m², every three weeks; 60-175 mg/m², weekly, without break; 20-150 mg/m² twice a week; and 150-250 mg/m² twice a week. The dosing frequency of the mTOR inhibitor nanoparticle composition (such as sirolimus/albumin nanoparticle composition) may be adjusted over the course of the treatment based on the judgment of the administering physician.

In some embodiments, the individual is treated for at least about any of one, two, three, four, five, six, seven, eight, nine, or ten treatment cycles.

The mTOR inhibitor nanoparticle composition (such as sirolimus/albumin nanoparticle composition) described herein allow infusion of the mTOR inhibitor nanoparticle composition to an individual over an infusion time that is shorter than about 24 hours. For example, in some embodiments, the mTOR inhibitor nanoparticle composition (such as sirolimus/albumin nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes.

In some embodiments, the exemplary dose of the mTOR inhibitor (in some embodiments a limus drug, e.g., sirolimus) in the mTOR inhibitor nanoparticle composition includes, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 160 mg/m², 175 mg/m², 200 mg/m², 210 mg/m², 220 mg/m², 260 mg/m², and 300 mg/m². For example, the dosage of an mTOR inhibitor (such as a limus drug, e.g., sirolimus or a derivative thereof) in a nanoparticle composition can be in the range of about 20-400 mg/m² when given on a 3-week schedule, or about 10-250 mg/m² when given on a weekly schedule.

In some embodiments, the dosage of an mTOR inhibitor (such as a limus drug, e.g., sirolimus) is about 100 mg to about 400 mg, for example about 100 mg, about 200 mg, about 300 mg, or about 400 mg. In some embodiments, the limus drug is administered at about 100 mg weekly, about 200 mg weekly, about 300 mg weekly, about 100 mg twice weekly, or about 200 mg twice weekly. In some embodiments, the administration is further followed by a monthly maintenance dose (which can be the same or different from the weekly doses).

In some embodiments when the mTOR nanoparticle composition is administered intravenously, the dosage of an mTOR inhibitor (such as a limus drug, e.g., sirolimus) in a nanoparticle composition can be in the range of about 30 mg to about 400 mg. The mTOR inhibitor nanoparticle composition (such as sirolimus/albumin nanoparticle composition) described herein allow infusion of the mTOR inhibitor nanoparticle composition to an individual over an infusion time that is shorter than about 24 hours. For example, in some embodiments, the mTOR inhibitor nanoparticle composition (such as sirolimus/albumin nanoparticle composition) is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes to about 40 minutes.

In some embodiments, the exemplary dosing schedules for the administration of an estrogen suppressor such as an agent that suppresses estrogen or estrogen receptor signaling is or is similar to the dosing schedule of such an agent when administered alone. For example, in some embodiments, the estrogen suppressor is letrozole and is administered daily. In some embodiments, letrozole is administered to the individual daily at an amount of about 0.1 mg to about 10 mg, such as 2.5 mg. In some embodiments, the estrogen suppressor, such as an agent that suppresses estrogen receptor signaling, is fulvestrant and is administered to the individual on days 1, 15, and 29 of an initiation cycle followed by monthly maintenance doses. In some embodiments, the estrogen suppressor, such as an agent that suppresses estrogen receptor signaling, is fulvestrant and is administered to the individual on days 1, 15, and 29 of an initiation cycle followed by monthly maintenance doses at an amount of about 100 mg to about 700 mg, such as 500 mg.

An mTOR inhibitor nanoparticle composition (such as sirolimus/albumin nanoparticle composition) and an estrogen suppressor (e.g., letrozole or fulvestrant), in pure form or in an appropriate pharmaceutical composition, can be administered via any of the accepted modes of administration or agents known in the art. The compositions and/or agents can be administered, for example, parenterally (such as intravenous). The dosage form can be, for example, a solid, semi-solid, lyophilized powder, or liquid dosage form, such as tablets, pills, soft elastic or hard gelatin capsules, powders, solutions, suspensions, suppositories, aerosols, or the like, preferably in unit dosage forms suitable for simple administration of precise dosages.

As discussed above, the mTOR inhibitor nanoparticle composition (such as sirolimus/albumin nanoparticle composition) and an estrogen suppressor (e.g., letrozole or fulvestrant) can be administered in a single unit dose or separate dosage forms. Accordingly, the phrase “pharmaceutical combination” includes a combination of two drugs in either a single dosage form or a separate dosage forms, i.e., the pharmaceutically acceptable carriers and excipients described throughout the application can be combined with an mTOR inhibitor nanoparticle composition (such as sirolimus/albumin nanoparticle composition) and an estrogen suppressor (e.g., letrozole or fulvestrant) in a single unit dose, as well as individually combined with an mTOR inhibitor nanoparticle composition and an estrogen suppressor (e.g., letrozole or fulvestrant) when these compounds are administered separately.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as citric acid, sorbitan monolaurate, triethanolamine oleate, butylated hydroxytoluene, and the like.

Solid dosage forms can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, or dispersing, the mTOR inhibitor nanoparticle composition (such as sirolimus/albumin nanoparticle composition) or estrogen suppressor (e.g., letrozole or fulvestrant) described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, such as ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

In some embodiments, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compound described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed., (Mack Publishing Company, Easton, Pa., 1990).

The mTOR inhibitor nanoparticle composition (such as sirolimus/albumin nanoparticle composition) can be administered to an individual (such as a human) via various routes, including, for example, via intravenous administration. In some embodiments, sustained continuous release formulation of the composition may be used. In some embodiments, the composition is administered intravenously.

The estrogen suppressor, such as an agent that suppresses estrogen or estrogen receptor signaling (e.g., letrozole or fulvestrant) can be administered to an individual (such as a human) via various routes, including, for example, oral, intramuscular, intravenous, intra-arterial, intraperitoneal, intrapulmonary, inhalation, intravesicular, intra-tracheal, subcutaneous, intraocular, intrathecal, transmucosal, and transdermal. In some embodiments, the estrogen suppressor is letrozole and is administered to the individual orally. In some embodiments, the estrogen suppressor is fulvestrant and is administered to the individual intramuscularly.

### C. Treatment of Hormone-Dependent Cancers

In some embodiments, provided is a method of treating a hormone-dependent cancer in an individual in need thereof, wherein the hormone-dependent cancer is selected from the group consisting of endometrial cancer (e.g., endometrioid endometrial cancer) and hormone receptor positive breast cancer, the method comprising administering to the individual: (a) a composition comprising nanoparticles comprising an mTOR inhibitor and an albumin; and (b) an estrogen suppressor, such as an agent that suppresses estrogen or estrogen receptor signaling (e.g., letrozole or fulvestrant).

Endometrioid endometrial cancer (EEC) is an endometrial cancer with origins in the endometrium of the uterus. Endometrioid endometrial cancer typically starts in gland cells of the endometrium. Endometrial cancers can be classified as estrogen-dependent type I and estrogen-independent class II. Type I endometrial tumors, also known as low grade endometrioid, make up the majority of endometrial cancer cases (~85%), are low grade with a glandular structure, usually express high levels of estrogen receptor α (ER), and are thought to be hormonally driven. Type II tumors include high-grade endometrioid tumors, serous tumors, clear cell tumors, carcinosarcomas, and tumors with mixed histology. The major subtypes of endometroid endometrial cancer are defined by genetic features of their cells, such as a mutation in the POLE gene, a high degree of “microsatellite instability,” microsatellite stability, and high copy number of certain sections of DNA. Examples of non-endometrioid endometrial cancer include papillary serous cancers, clear cell tumors, and carcinosarcomas. EECs are usually cystic and solid tumors with foci of necrosis and hemorrhage. EECs are morphologically similar to the usual type of adenocarcinoma of the endometrium, and they are graded using the same criteria. Squamous differentiation may be seen in EEC. There are a number of EEC subtypes, including adenocarcinomas (such as having squamous differentiation), adenoacanthoma, adenosquamous (or mixed cell), secretory carcinoma, ciliated carcinoma, and villoglandular adenocarcinoma. In some embodiments, the EEC is estrogen-dependent EEC. Techniques for determining hormone dependency and hormone receptor presence are known in the art, e.g., obtaining a biopsy to test for, e.g., the presence of estrogen or progesterone receptor using techniques such as immunohistochemistry (IHC).

In some embodiments, there is provided a method of treating endometrial cancer (e.g., endometrioid endometrial cancer) in an individual (such as a human) comprising administering to the individual: (a) a composition comprising nanoparticles comprising an mTOR inhibitor (such as a limus drug, e.g., sirolimus or a derivative thereof) and an albumin; and (b) an estrogen suppressor (e.g., letrozole). In some embodiments, the method comprises administering to the individual: (a) a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and (b) an estrogen suppressor (e.g., letrozole). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and (b) an estrogen suppressor (e.g., letrozole). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and (b) an estrogen suppressor (e.g., letrozole). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and (b) an estrogen suppressor (e.g., letrozole). In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the estrogen suppressor is letrozole. In some embodiments, the mTOR inhibitor nanoparticle composition is administered weekly, such as twice out of every 3 weeks, such as on days 1 and 8, 1 and 15, 1 and 21, or 8 and 15 of a 21-day cycle. In some embodiments, the amount of the mTOR inhibitor in the mTOR inhibitor nanoparticle composition is about 10 mg/m² to about 150 mg/m², such as about any of 100 mg/m², 75 mg/m², 56 mg/m², 45 mg/m², or 30 mg/m². In some embodiments, the mTOR inhibitor nanoparticle composition is administered intravenously. In some embodiments, the mTOR inhibitor nanoparticle composition is administered over an infusion period of less than about any of 24 hours, 12 hours, 8 hours, 5 hours, 3 hours, 2 hours, 1 hour, 30 minutes, 20 minutes, or 10 minutes. In some embodiments, the mTOR inhibitor nanoparticle composition (such as sirolimus/albumin nanoparticle composition) is administered over an infusion period of about 30 minutes. In some embodiments, the estrogen suppressor (e.g., letrozole) is administered daily. In some embodiments, the amounts of the estrogen suppressor (e.g., letrozole) is about 0.1 mg to about 10 mg, such as about 2.5 mg. In some embodiments, the estrogen suppressor (e.g., letrozole) is administered orally. In some embodiments, the estrogen suppressor (e.g., letrozole) is orally administered daily at about 0.1 mg to about 10 mg, including about 2.5 mg. In some embodiments, there is provided a method of treating endometrial cancer (e.g., endometrioid endometrial cancer) in an individual (such as a human) comprising: (a) intravenously administering to the individual a composition comprising nanoparticles comprising sirolimus and an albumin weekly two out of every three weeks (e.g., on day 1 and 8 of a 21-day cycle) at an amount of about 10 mg/m² to about 150 mg/m², such as about 100 mg/m²; and (b) orally administering to the individual letrozole daily at an amount of about 0.1 mg to about 10 mg, such as about 2.5 mg. In some embodiments, the endometrial cancer (e.g., endometrioid endometrial cancer) is advanced (unresectable International Federation of Gynecology and Obstetrics (FIGO) Stage III or IV) or recurrent endometrial cancer (e.g., endometrioid endometrial cancer).

In some embodiments, the endometrial cancer (e.g., endometrioid endometrial cancer) is refractory, relapsed, recurrent, or resistant to a prior treatment. In some embodiments, the prior treatment comprises a chemotherapy. In some embodiments, the prior treatment comprises a platinum agent, immunotherapy, targeted therapy, and/or checkpoint inhibitor. In some embodiments, the prior treatment comprises a platinum agent and/or a checkpoint inhibitor. In some embodiments, the individual has received zero or 1 or more prior treatments, such as 0-1 or more chemotherapy regimens. In some embodiments, the individual has cancer that is recurrent, advanced, or metastatic. In some embodiments, the individual has received 1 or more lines of chemotherapy. In some embodiments, the individual has received 0 line of chemotherapy, or is chemotherapy-naïve. In some embodiments, the prior therapy is an adjuvant therapy. In some embodiments, the prior adjuvant therapy is a chemotherapy, a hormonal therapy, or a checkpoint inhibitor. In some embodiments, the adjuvant therapy is completed ≥6 months prior to treatment according to the method described herein. In some embodiments, the prior therapy is a non-chemotherapy-based treatment. In some embodiments, the prior non-chemotherapy-based ended ≥4 months prior to treatment according to the method described herein.

In some embodiments, the individual has not been previously administered a treatment for a cancer, such as a cancer being treated by the methods described herein. In some embodiments, the cancer is endometrial cancer. In some embodiments, the individual has cancer that is recurrent, advanced, or metastatic. In some embodiments, the individual has not been previously administered a chemotherapy. In some embodiments, the prior treatment comprises a platinum agent, immunotherapy, targeted therapy, and/or checkpoint inhibitor. In some embodiments, the individual has not been previously administered a platinum agent and/or a checkpoint inhibitor. In some embodiments, the individual has not been previously administered an adjuvant therapy. In some embodiments, the individual has not been previously administered a non-chemotherapy-based treatment.

Hormone receptor positive breast cancers are breast cancer expressing estrogen receptors, progesterone receptors, or both estrogen and progesterone receptors. Presence of such receptors serve to stimulate growth in the presence of estrogen and/or progesterone (dependent on the expressed receptors). In some embodiments, hormone receptor positive breast cancers may have other clinically relevant markers, such as HER2. Techniques for determining hormone dependency and hormone receptor presence are known in the art, e.g., obtaining a biopsy to test for, e.g., the presence of estrogen or progesterone receptor using techniques such as immunohistochemistry (IHC).

In some embodiments, there is provided a method of treating hormone receptor positive breast cancer in an individual (such as a human) comprising administering to the individual: (a) a composition comprising nanoparticles comprising an mTOR inhibitor (such as a limus drug, e.g., sirolimus or a derivative thereof) and an albumin; and (b) an estrogen suppressor (e.g., fulvestrant). In some embodiments, the method comprises administering to the individual: (a) a composition comprising nanoparticles comprising an mTOR inhibitor (such as a limus drug, e.g., sirolimus or a derivative thereof) and an albumin, wherein the mTOR inhibitor in the nanoparticles is associated (e.g., coated) with the albumin; and (b) an estrogen suppressor (e.g., fulvestrant). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles have an average particle size of no greater than about 150 nm (such as no greater than about 120 nm); and (b) an estrogen suppressor (e.g., fulvestrant). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with albumin, wherein the nanoparticles have an average particle size of no greater than about 150 nm (such as no greater than about 120 nm); and (b) an estrogen suppressor (e.g., fulvestrant). In some embodiments, the method comprises administering to the individual: (a) a composition comprising nanoparticles comprising an mTOR inhibitor (such as a limus drug, e.g., sirolimus or a derivative thereof) and an albumin, wherein the nanoparticles comprise the mTOR inhibitor associated (e.g., coated) with the albumin, wherein the nanoparticles have an average particle size of no greater than about 150 nm (such as no greater than about 120 nm, for example about 100 nm), wherein the weight ratio of albumin and the mTOR inhibitor in the mTOR inhibitor nanoparticle composition is about 9:1 or less (such as about 9:1 or about 8:1); and (b) an estrogen suppressor (e.g., fulvestrant). In some embodiments, the mTOR inhibitor is a limus drug. In some embodiments, the mTOR inhibitor is sirolimus or a derivative thereof. In some embodiments, the mTOR inhibitor nanoparticle composition comprises nab-sirolimus. In some embodiments, the mTOR inhibitor nanoparticle composition is nab-sirolimus. In some embodiments, the estrogen suppressor is fulvestrant. In some embodiments, the mTOR inhibitor nanoparticle composition is administered weekly, such as twice out of every 3 weeks, such as on days 1 and 8, 1 and 15, 1 and 21, or 8 and 15 of a 21-day cycle. In some embodiments, the amount of the mTOR inhibitor in the mTOR inhibitor nanoparticle composition is about 10 mg/m² to about 150 mg/m², such as about any of 100 mg/m², 75 mg/m², 56 mg/m², 45 mg/m², or 30 mg/m². In some embodiments, the mTOR inhibitor nanoparticle composition is administered intravenously. In some embodiments, the estrogen suppressor (e.g., fulvestrant) is administered using an initiation cycle followed by one or more maintenance cycle (e.g., administering fulvestrant to the individual on days 1, 15, and 29 of an initiation cycle followed by one or more monthly maintenance doses). In some embodiments, the amount of the estrogen suppressor (e.g., fulvestrant) is about 100 mg to about 700 mg, such as 500 mg. In some embodiments, the estrogen suppressor (e.g., fulvestrant) is administered intramuscularly. In some embodiments, the estrogen suppressor (e.g., fulvestrant) is intramuscularly administered using an initiation cycle followed by one or more maintenance cycles at about 100 mg to about 700 mg, such as 500 mg. In some embodiments, there is provided a method of treating hormone receptor positive breast cancer in an individual (such as a human) comprising: (a) intravenously administering to the individual a composition comprising nanoparticles comprising sirolimus and an albumin weekly two out of every three weeks (e.g., on day 1 and 8 of a 21-day cycle) at an amount of about 10 mg/m² to about 150 mg/m², such as about 100 mg/m²; and (b) intramuscularly administering to the individual fulvestrant using an initiation cycle followed by one or more maintenance cycles at about 100 mg to about 700 mg, such as 500 mg.

### III. Compositions Comprising Nanoparticles Comprising an mTOR Inhibitor

The mTOR inhibitor nanoparticle compositions described herein comprise nanoparticles comprising (in various embodiments consisting essentially of or consisting of) an mTOR inhibitor (such as a limus drug, e.g., sirolimus or a derivative thereof) and an albumin (such as human serum albumin). It is noted that the terms sirolimus and rapamycin are used interchangeably herein. Nanoparticles of poorly water soluble drugs (such as macrolides) have been disclosed in, for example, U.S. Pat. Nos. 5,916,596; 6,506,405; 6,749,868, 6,537,579, 7,820,788, and 8,911,786, 11,497,737, and also in U.S. Pat. Pub. Nos. 2006/0263434, and 2007/0082838; PCT Patent Application WO08/137148, U.S. Patent Application No. 62/927,047, each of which is incorporated herein by reference in their entirety.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 10 to about 400 nm. In some embodiments, the average or mean diameter of the nanoparticles is about 10 to about 150 nm. In some embodiments, the average or mean diameter of the nanoparticles is about 40 to about 120 nm. In some embodiments, the average or mean diameter of the nanoparticles are no less than about 50 nm. In some embodiments, the nanoparticles are sterile-filterable.

Methods of determining average particle sizes are known in the art, for example, dynamic light scattering (DLS) has been routinely used in determining the size of submicrometre-sized particles based. International Standard ISO22412 Particle Size Analysis—Dynamic Light Scattering, International Organisation for Standardisation (ISO) 2008 and Dynamic Light Scattering Common Terms Defined, Malvern Instruments Limited, 2011. In some embodiments, the particle size is measured as the volume-weighted mean particle size (Dv50) of the nanoparticles in the composition.

In some embodiments, the nanoparticles comprise the mTOR inhibitor associated with the albumin. In some embodiments, the nanoparticles comprise the mTOR inhibitor coated with the albumin.

In some embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprising the mTOR inhibitor (such as a limus drug, e.g., sirolimus or a derivative thereof) are associated (e.g., coated) with an albumin (such as human albumin or human serum albumin). In some embodiments, the composition comprises an mTOR inhibitor (such as a limus drug, e.g., rapamycin or a derivative thereof) in both nanoparticle and non-nanoparticle forms (e.g., in the form of solutions or in the form of soluble albumin/nanoparticle complexes), wherein at least about any one of 50%, 60%, 70%, 80%, 90%, 95%, or 99% of the mTOR inhibitor in the composition are in nanoparticle form. In some embodiments, the mTOR inhibitor (such as a limus drug, e.g., rapamycin or a derivative thereof) in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of an mTOR inhibitor (such as a limus drug, e.g., rapamycin or a derivative thereof) that is substantially free of polymeric materials (such as polymeric matrix).

In some embodiments, the composition comprises an albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of the albumin to the mTOR inhibitor (such as a limus drug, e.g., rapamycin or a derivative thereof) in the mTOR inhibitor nanoparticle composition is such that a sufficient amount of mTOR inhibitor binds to, or is transported by, the cell. While the weight ratio of an albumin to an mTOR inhibitor (such as a limus drug, e.g., rapamycin or a derivative thereof) will have to be optimized for different albumin and mTOR inhibitor combinations, generally the weight ratio of an albumin to an mTOR inhibitor (such as a limus drug, e.g., rapamycin or a derivative thereof) (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to mTOR inhibitor (such as a limus drug, e.g., rapamycin or a derivative thereof) weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human albumin or human serum albumin) to the mTOR inhibitor (such as a limus drug, e.g., rapamycin or a derivative thereof) in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the composition comprises nanoparticles comprising an mTOR inhibitor and an albumin, wherein the weight ratio of the albumin to the mTOR inhibitor in the composition is about 0.01:1 to about 100:1. In some embodiments, the composition comprises nanoparticles comprising an mTOR inhibitor (such as rapamycin) and an albumin, wherein the weight ratio of the albumin to the mTOR inhibitor (such as rapamycin) in the composition is about 18:1 or less (including for example any of about 1:1 to about 18:1, about 2:1 to about 15:1, about 3:1 to about 12:1, about 4:1 to about 10:1, about 5:1 to about 9:1, and about 9:1). In some embodiments, the composition comprises nanoparticles comprising rapamycin, or a derivative thereof, and an albumin, wherein the weight ratio of the albumin to the rapamycin or derivative thereof in the composition is about 18:1 or less (including for example any of about 1:1 to about 18:1, about 2:1 to about 15:1, about 3:1 to about 12:1, about 4:1 to about 10:1, about 5:1 to about 9:1, and about 9:1). In some embodiments, the mTOR inhibitor (such as rapamycin) is coated with albumin.

In some embodiments, the mTOR inhibitor nanoparticle composition (such as rapamycin/albumin nanoparticle composition) comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises an albumin (such as human albumin or human serum albumin). The albumin may either be natural in origin or synthetically prepared. In some embodiments, the albumin is human albumin or human serum albumin. In some embodiments, the albumin is a recombinant albumin.

Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulfide bridges, one free thiol (Cys 34), and a single tryptophan (Trp 214). Intravenous use of HSA solution has been indicated for the prevention and treatment of hypovolemic shock (see, e.g., Tullis, JAMA, 237: 355-360, 460-463, (1977)) and Houser et al., Surgery, Gynecology and Obstetrics, 150: 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context). Human serum albumin (HSA) has multiple hydrophobic binding sites (a total of eight for fatty acids, an endogenous ligand of HSA) and binds a diverse set of drugs, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Rapamycin and propofol have been shown to bind HSA (see, e.g., Paal et al., Eur. J. Biochem., 268(7), 2187-91 (200a), Purcell et al., Biochem. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

An mTOR inhibitor (such as a limus drug, e.g., rapamycin or a derivative thereof) is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as a human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed using an optical microscope at 1000 times, at about fifteen minutes after preparation of the suspension. Stability can also be evaluated under accelerated testing conditions, such as at a temperature that is about 40° C. or higher.

The compositions described herein may be a stable aqueous suspension of the mTOR inhibitor, such as a stable aqueous suspension of the mTOR inhibitor at a concentration of any of about 0.1 to about 200 mg/ml, about 0.1 to about 150 mg/ml, about 0.1 to about 100 mg/ml, about 0.1 to about 50 mg/ml, about 0.1 to about 20 mg/ml, about 1 to about 10 mg/ml, about 2 mg/ml to about 8 mg/ml, about 4 to about 6 mg/ml, and about 5 mg/ml. In some embodiments, the concentration of the mTOR inhibitor is at least about any of 0.2 mg/ml, 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, 50 mg/ml, 100 mg/ml, 150 mg/ml, or 200 mg/ml.

In some embodiments, the albumin is present in an amount that is sufficient to stabilize the mTOR inhibitor (such as a limus drug, e.g., rapamycin or a derivative thereof) in an aqueous suspension at a certain concentration. For example, the concentration of the mTOR inhibitor (such as a limus drug, e.g., rapamycin or a derivative thereof) in the composition is about 0.1 to about 100 mg/ml, including for example about any of 0.1 to about 50 mg/ml, about 0.1 to about 20 mg/ml, about 1 to about 10 mg/ml, about 2 mg/ml to about 8 mg/ml, about 4 to about 6 mg/ml, or about 5 mg/ml. In some embodiments, the concentration of the mTOR inhibitor (such as a limus drug, e.g., rapamycin or a derivative thereof) is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g., about 0.5% (w/v), about 5% (w/v), about 10% (w/v), about 15% (w/v), about 20% (w/v), about 30% (w/v), about 40% (w/v), or about 50% (w/v)) of an albumin. In some embodiments, the composition, in liquid form, comprises about 0.5% to about 5% (w/v) of albumin.

In some embodiments, the albumin allows the composition to be administered to an individual (such as a human) without significant side effects. In some embodiments, the albumin (such as human serum albumin or human albumin) is in an amount that is effective to reduce one or more side effects of administration of the mTOR inhibitor (such as a limus drug, e.g., rapamycin or a derivative thereof) to a human. The term “reducing one or more side effects” of administration of the mTOR inhibitor (such as a limus drug, e.g., rapamycin or a derivative thereof) refers to reduction, alleviation, elimination, or avoidance of one or more undesirable effects caused by the mTOR inhibitor, as well as side effects caused by delivery vehicles (such as solvents that render the limus drugs suitable for injection) used to deliver the mTOR inhibitor. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with limus drugs (such as a limus drug, e.g., rapamycin or a derivative thereof) can be reduced.

In some embodiments, the composition is a dry (such as lyophilized) composition that can be reconstituted, resuspended, or rehydrated to form generally a stable aqueous suspension of the nanoparticles comprising an mTOR inhibitor and an albumin. In some embodiments, the composition is a liquid (such as aqueous) composition obtained by reconstituting or resuspending a dry composition. In some embodiments, the composition is an intermediate liquid (such as aqueous) composition that can be dried (such as lyophilized).

A. mTOR Inhibitors

The methods described herein in some embodiments comprise administration of nanoparticle compositions of mTOR inhibitors. “mTOR inhibitor” used herein refers to an inhibitor of mTOR. mTOR is a serine/threonine-specific protein kinase downstream of the phosphatidylinositol 3-kinase (PI3K)/Akt (protein kinase B) pathway, and a key regulator of cell survival, proliferation, stress, and metabolism. mTOR pathway dysregulation has been found in many human carcinomas, and mTOR inhibition produced substantial inhibitory effects on tumor progression.

The mammalian target of rapamycin (mTOR) (also known as mechanistic target of rapamycin or FK506 binding protein 12-rapamycin associated protein 1 (FRAP1)) is an atypical serine/threonine protein kinase that is present in two distinct complexes, mTOR Complex 1 (mTORC1) and mTOR Complex 2 (mTORC2). mTORC1 is composed of mTOR, regulatory-associated protein of mTOR (Raptor), mammalian lethal with SEC13 protein 8 (MLST8), PRAS40 and DEPTOR (Kim et al. (2002). Cell 110: 163-75; Fang et al. (2001). Science 294 (5548): 1942-5). mTORC1 integrates four major signal inputs: nutrients (such as amino acids and phosphatidic acid), growth factors (insulin), energy and stress (such as hypoxia and DNA damage). Amino acid availability is signaled to mTORC1 via a pathway involving the Rag and Ragulator (LAMTOR1-3) Growth factors and hormones (e.g., insulin) signal to mTORC1 via Akt, which inactivates TSC2 to prevent inhibition of mTORC1. Alternatively, low ATP levels lead to the AMPK-dependent activation of TSC2 and phosphorylation of raptor to reduce mTORC1 signaling proteins.

Active mTORC1 has a number of downstream biological effects including translation of mRNA via the phosphorylation of downstream targets (4E-BP1 and p70 S6 Kinase), suppression of autophagy (Atg13, ULK1), ribosome biogenesis, and activation of transcription leading to mitochondrial metabolism or adipogenesis. Accordingly, mTORC1 activity promotes either cellular growth when conditions are favorable or catabolic processes during stress or when conditions are unfavorable.

mTORC2 is composed of mTOR, rapamycin-insensitive companion of mTOR (RICTOR), GβL, and mammalian stress-activated protein kinase interacting protein 1 (mSIN1). In contrast to mTORC1, for which many upstream signals and cellular functions have been defined (see above), relatively little is known about mTORC2 biology. mTORC2 regulates cytoskeletal organization through its stimulation of F-actin stress fibers, paxillin, RhoA, Rac1, Cdc42, and protein kinase Cα (PKCα). It had been observed that knocking down mTORC2 components affects actin polymerization and perturbs cell morphology (Jacinto et al. (2004). Nat. Cell Biol. 6, 1122-1128; Sarbassov et al. (2004). Curr. Biol. 14, 1296-1302). This suggests that mTORC2 controls the actin cytoskeleton by promoting protein kinase Cα (PKCα) phosphorylation, phosphorylation of paxillin and its relocalization to focal adhesions, and the GTP loading of RhoA and Rac1. The molecular mechanism by which mTORC2 regulates these processes has not been determined.

In some embodiments, the mTOR inhibitor (such as a limus drug, e.g., sirolimus or a derivative thereof) is an inhibitor of mTORC1. In some embodiments, the mTOR inhibitor (such as a limus drug, e.g., sirolimus or a derivative thereof) is an inhibitor of mTORC2. In some embodiments, the mTOR inhibitor (such as a limus drug, e.g., sirolimus or a derivative thereof) is an inhibitor of both mTORC1 and mTORC2.

In some embodiments, the mTOR inhibitor is a limus drug, which includes sirolimus and its analogs. Examples of limus drugs include, but are not limited to, temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the limus drug is selected from the group consisting of temsirolimus (CCI-779), everolimus (RAD001), ridaforolimus (AP-23573), deforolimus (MK-8669), zotarolimus (ABT-578), pimecrolimus, and tacrolimus (FK-506). In some embodiments, the mTOR inhibitor is an mTOR kinase inhibitor, such as CC-115 or CC-223.

In some embodiments, the mTOR inhibitor is sirolimus. Sirolimus is macrolide antibiotic that complexes with FKBP-12 and inhibits the mTOR pathway by binding mTORC1.

In some embodiments, the mTOR inhibitor is selected from the group consisting of sirolimus (rapamycin), BEZ235 (NVP-BEZ235), everolimus (also known as RAD001, Zortress, Certican, and Afinitor), AZD8055, temsirolimus (also known as CCI-779 and Torisel), CC-115, CC-223, PI-103, Ku-0063794, INK 128, AZD2014, NVP-BGT226, PF-04691502, CH5132799, GDC-0980 (RG7422), Torin 1, WAY-600, WYE-125132, WYE-687, GSK2126458, PF-05212384 (PKI-587), PP-121, OSI-027, Palomid 529, PP242, XL765, GSK1059615, WYE-354, and ridaforolimus (also known as deforolimus).

BEZ235 (NVP-BEZ235) is an imidazoquinoline derivative that is an mTORC1 catalytic inhibitor (Roper J, et al. PLoS One, 2011, 6(9), e25132). Everolimus is the 40-O-(2-hydroxyethyl) derivative of sirolimus and binds the cyclophilin FKBP-12, and this complex also mTORC1. AZD8055 is a small molecule that inhibits the phosphorylation of mTORC1 (p70S6K and 4E-BP1). Temsirolimus is a small molecule that forms a complex with the FK506-binding protein and prohibits the activation of mTOR when it resides in the mTORC1complex. PI-103 is a small molecule that inhibits the activation of the rapamycin-sensitive (mTORC1) complex (Knight et al. (2006) Cell. 125: 733-47). KU-0063794 is a small molecule that inhibits the phosphorylation of mTORC1 at Ser2448 in a dose-dependent and time-dependent manner. INK 128, AZD2014, NVP-BGT226, CH5132799, WYE-687, and are each small molecule inhibitors of mTORC1. PF-04691502 inhibits mTORC1 activity. GDC-0980 is an orally bioavailable small molecule that inhibits Class I PI3 Kinase and TORC1. Torin 1 is a potent small molecule inhibitor of mTOR. WAY-600 is a potent, ATP-competitive and selective inhibitor of mTOR. WYE-125132 is an ATP-competitive small molecule inhibitor of mTORC1. GSK2126458 is an inhibitor of mTORC1. PKI-587 is a highly potent dual inhibitor of PI3Kα, PI3Kγ and mTOR. PP-121 is a multi-target inhibitor of PDGFR, Hck, mTOR, VEGFR2, Src and Abl. OSI-027 is a selective and potent dual inhibitor of mTORC1 and mTORC2 with IC50 of 22 nM and 65 nM, respectively. Palomid 529 is a small molecule inhibitor of mTORC1 that lacks affinity for ABCB1/ABCG2 and has good brain penetration (Lin et al. (2013) Int J Cancer DOI: 10.1002/ijc. 28126 (e-published ahead of print). PP242 is a selective mTOR inhibitor. XL765 is a dual inhibitor of mTOR/PI3k for mTOR, p110α, p110β, p110γ and p110δ. GSK1059615 is a novel and dual inhibitor of PI3Kα, PI3Kβ, PI3Kδ, PI3Kγ and mTOR. WYE-354 inhibits mTORC1 in HEK293 cells (0.2 μM-5 μM) and in HUVEC cells (10 nM-1 μM). WYE-354 is a potent, specific, and ATP-competitive inhibitor of mTOR. Deforolimus (Ridaforolimus, AP23573, MK-8669) is a selective mTOR inhibitor.

### B. Other Components in the Nanoparticle Composition

In some embodiments, the composition is suitable for administration to a human. In some embodiments, the composition is suitable for administration to a mammal such as, in the veterinary context, domestic pets and agricultural animals. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl- and propylhydroxybenzoates, talc, magnesium stearate, and mineral oil. The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. Injectable formulations are preferred.

In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

### C. Albumin-Based Nanoparticle Compositions of Sirolimus

The methods described herein are particularly suitable for albumin-based nanoparticle compositions described herein in more details. The nanoparticle composition in some embodiments includes (a) nanoparticles that include rapamycin and albumin, and (b) a non-nanoparticle portion that includes rapamycin and albumin. The rapamycin and the albumin of the nanoparticles are associated with each other in the nanoparticles. For example, the nanoparticles may include a coating having the albumin, which surrounds a core comprising the rapamycin. In the non-nanoparticle portion of the composition, the rapamycin and the albumin may or may not associated with each other (i.e., the rapamycin may be in a reversible binding equilibrium with the albumin), but do not associate with each other in a manner that forms nanoparticles. That is, the nanoparticle composition may include nanoparticle-bound albumin and nanoparticle-bound rapamycin in the nanoparticle portion of the composition, and non-nanoparticle albumin and non-nanoparticle rapamycin in the non-nanoparticle portion of the composition. As used herein, “in the nanoparticles” is used synonymously with “in the nanoparticle portion.” The albumin of the nanoparticles may be further distinguishable from the albumin in the non-nanoparticle portion of the composition; for example, the oligomeric profile of the albumin in the nanoparticles may differ from the oligomeric profile of the albumin in the non-nanoparticle portion of the composition. The oligomer profile means the percentage of various albumin species compared with the total albumin in the composition. The types of albumin species includes albumin monomers, dimers, trimers, oligomers, and polymers. As used herein, “albumin monomers” or “monomeric albumin” refers to an albumin species having one, and only one, albumin unit; “albumin dimers” or “dimeric albumin” refers to an albumin species having two, and only two, albumin units; “albumin trimers” or “trimeric albumin” refers to albumin species having three, and only three, albumin units; “albumin polymers” refers to albumin species having a higher molecular weight than albumin monomers and albumin dimers; “albumin oligomers” or “oligomeric albumin” refers to lower molecular weight polymeric albumin species associated with a UV-based size-exclusion chromatography peak observed between a peak associated with albumin dimers and higher molecular weight polymeric albumin species.

The albumin of the nanoparticles associates with the rapamycin of the nanoparticles so that a nanoparticle suspension has a high concentration of rapamycin, which allows the composition to be used as a pharmaceutical composition for treating certain diseases, such as cancer. Manufactured nanoparticles (which may be made, for example, using the methods described herein) may be formulated, filtered, or otherwise processed to obtain the pharmaceutical composition, which may be suitable for medical use in a human individual.

Generally, to make the rapamycin pharmaceutical compositions described herein, rapamycin is dissolved in an organic solvent. Suitable organic solvents include, for example, ketones, esters, ethers, chlorinated solvents, and other solvents known in the art. For example, the organic solvent can be a mixture of methylene chloride/ethanol, chloroform/ethanol, or chloroform/tert-butanol (for example with a ratio of about any one of 1:9, 1:8, 1:7, 1:6, 1:5, 1:4, 1:3, 1:2, 1:1, 2:1, 3:1, 4:1, 5:1, 6:1, 7:1, 8:1, or 9:1 or with a ratio of about any one of 3:7, 5:7, 4:6, 5:5, 6:5, 8:5, 9:5, 9.5:5, 5:3, 7:3, 6:4, or 9.5:0.5). In some embodiments, the organic solvent comprises between about 10% and about 50% tert-butanol by volume. In some embodiments, the organic solvent comprises about any of 10%, 15%, 20%, 25%, 30%, 35%, 40%, 45%, or 50% tert-butanol by volume. In some embodiments, the organic solvent comprises about any of 10-15%, 15-20%, 20-25%, 25-30%, 30-35%, 35-40%, 40-45%, or 45-50%, or any combination of such ranges, of tert-butanol by volume. In some embodiments, the organic solvent comprises between about 50% and about 90% chloroform by volume. In some embodiments, the organic solvent comprises about any of 50%, 55%, 60%, 65%, 70%, 75%, 80%, 85%, or 90% chloroform by volume. In some embodiments, the organic solvent comprises about any of 50-55%, 55-60%, 60-65%, 65-70%, 70-75%, 75-80%, 80-85%, or 85-90%, or any combination of such ranges, of chloroform by volume. In some embodiments, the organic solvent comprises between about 10% and about 50% tert-butanol by volume and between about 50% and about 90% chloroform by volume. In some embodiments, the organic solvent comprises chloroform and tert-butanol at a volumetric ratio of about 1:1 to about 1:9, such as about any of 1:1, 2:1, 3:1, 4:1, 5:1, 6:1, 7:1, 8:1, and 9:1.

Albumin (such as recombinant albumin, for example NOVOZYME™ recombinant albumin or INTRIVIA™ recombinant albumin disclosed herein) is dissolved in an aqueous solution (such as water) and combined with the rapamycin solution to form a crude emulsion. The mixture is subjected to high pressure homogenization (e.g., using an Avestin, APV Gaulin, MICROFLUIDIZER™ such as a MICROFLUIDIZER™ Processor M-110EH from Microfluidics, Stansted, or Ultra Turrax homogenizer). The emulsion may be cycled through the high pressure homogenizer for between about 2 to about 100 cycles, such as about 5 to about 50 cycles or about 6 to about 20 cycles (e.g., about any one of 6, 8, 10, 12, 14, 16, 18 or 20 cycles). The organic solvent can then be removed by evaporation utilizing suitable equipment known for this purpose, including, but not limited to, rotary evaporators, falling film evaporators, wiped film evaporators, spray driers, and the like that can be operated in batch mode or in continuous operation. In some embodiments, the evaporator is a wiped film evaporator. The solvent may be removed at reduced pressure (such as at about any one of 25 mm Hg, 30 mm Hg, 40 mm Hg, 50 mm Hg, 100 mm Hg, 200 mm Hg, or 300 mm Hg). The amount of time used to remove the solvent under reduced pressure may be adjusted based on the volume of the formulation. For example, for a formulation produced on a 300 mL scale, the solvent can be removed at about 1 to about 300 mm Hg (e.g., about any one of 5-100 mm Hg, 10-50 mm Hg, 20-40 mm Hg, or 25 mm Hg) for about 5 to about 60 minutes (e.g., about any one of 7, 8, 9, 10, 11, 12, 13, 14, 15 16, 18, 20, 25, or 30 minutes). The dispersion obtained can be further lyophilized.

The nanoparticle compositions described herein (such a pharmaceutical composition) may have distinct characteristics for any one or more (in any combination) of the following: (1) the oligomeric status of the albumin associated with (such as in) the nanoparticles, such as the percentage of albumin monomers, dimers, and/or polymers (or trimers) of the albumin associated with (such as in) the nanoparticles; (2) the oligomeric status of the albumin associated with (such as in) the non-nanoparticle portion of the composition, such as the percentage of albumin monomers, dimers, and/or polymers (or trimers) of the albumin associated with (such as in) the non-nanoparticle portion of the composition; (3) the oligomeric status of the total albumin in the composition, such as the percentage of albumin monomers, dimers, and/or polymers (or trimers) of the total albumin in the composition; (4) the particle size profile of the nanoparticles, such as the average particle size, polydispersity index, and/or size distribution; (5) the portion (e.g., weight percentage) of the nanoparticles that is albumin and/or the portion (e.g., weight percentage) of the nanoparticles that is rapamycin; (6) the weight ratio of the albumin to the rapamycin in the nanoparticles; (7) the weight ratio of the albumin to the rapamycin in the non-nanoparticle portion of the composition; (8) the weight ratio of the albumin to the rapamycin in the non-nanoparticle portion of the composition (9) the weight ratio of the total albumin to the total rapamycin in the composition; (10) the portion (e.g., weight percentage) of rapamycin that is in the nanoparticles (or the non-nanoparticle portion of the composition) compared to the total rapamycin in the composition; (11) the portion (e.g., weight percentage) of albumin that is in the non-nanoparticle portion (or in the nanoparticles) compared to the total albumin in the composition; (12) the concentration of albumin in the composition; (13) the concentration of albumin in the non-nanoparticle portion of the composition; (14) the concentration of albumin in the composition that is associated with (such as in) the nanoparticles; (15) the concentration of rapamycin in the composition; (16) the concentration of rapamycin in the non-nanoparticle portion of the composition; (17) the concentration of rapamycin in the composition that is associated with (such as in) the nanoparticles; (18) the osmolality of the composition; (19) the viscosity of the composition; (20) the pH of the composition; (21) the stability of the nanoparticles in the composition; (22) the amount of residual solvent in the composition; (23) the zeta potential of the nanoparticles in the composition; (24) the crystalline status of the rapamycin in the nanoparticles; (25) the particle morphology of the nanoparticles, such as the shape, sphericity, thickness of the coating, and/or surface-to-volume ratio; (26) the weight percentage of seco-rapamycin in the nanoparticles, as compared to the sum of seco-rapamycin and rapamycin, by weight; (27) the presence, percentage, or concentration of albumin stabilizer (such as sodium caprylate and/or N-acetyltryptophanate) in the composition; (28) the recovery of rapamycin following filtration; (29) in vitro release kinetics of the nanoparticles; (30) the portion of total rapamycin in the composition that is both in the non-nanoparticle portion of the composition and not bound to albumin; and/or (31) the weight percentage of seco-rapamycin in the composition, as compared to the sum of seco-rapamycin and rapamycin, by weight. In some embodiments, the oligomeric status (such as the percentage of albumin monomers, dimers, or polymers (or trimers)) of the nanoparticles, the non-nanoparticles portion, or the total composition is assessed by size-exclusion chromatography using a saline mobile phase coupled with a multiple angle light scattering (MALS) detector).

The nanoparticle compositions described herein (such a pharmaceutical composition) may have distinct characteristics for any one or more (in any combination) of the following: (1) the oligomeric status of the albumin associated with (such as in) the nanoparticles, such as the percentage of albumin monomers, dimers, oligomers, and/or polymers (other than oligomers) of the albumin associated with (such as in) the nanoparticles; (2) the oligomeric status of the albumin associated with (such as in) the non-nanoparticle portion of the composition, such as the percentage of albumin monomers, dimers, oligomers, and/or polymers (other than oligomers) of the albumin associated with (such as in) the non-nanoparticle portion of the composition; (3) the oligomeric status of the total albumin in the composition, such as the percentage of albumin monomers, dimers, oligomers, and/or polymers (other than oligomers) of the total albumin in the composition; (4) the particle size profile of the nanoparticles, such as the average particle size, polydispersity index, and/or size distribution; (5) the portion (e.g., weight percentage) of the nanoparticles that is albumin and/or the portion (e.g., weight percentage) of the nanoparticles that is rapamycin; (6) the weight ratio of the albumin to the rapamycin in the nanoparticles; (7) the weight ratio of the albumin to the rapamycin in the non-nanoparticle portion of the composition; (8) the weight ratio of the albumin to the rapamycin in the non-nanoparticle portion of the composition (9) the weight ratio of the total albumin to the total rapamycin in the composition; (10) the portion (e.g., weight percentage) of rapamycin that is in the nanoparticles (or the non-nanoparticle portion of the composition) compared to the total rapamycin in the composition; (11) the portion (e.g., weight percentage) of albumin that is in the non-nanoparticle portion (or in the nanoparticles) compared to the total albumin in the composition; (12) the concentration of albumin in the composition; (13) the concentration of albumin in the non-nanoparticle portion of the composition; (14) the concentration of albumin in the composition that is associated with (such as in) the nanoparticles; (15) the concentration of rapamycin in the composition; (16) the concentration of rapamycin in the non-nanoparticle portion of the composition; (17) the concentration of rapamycin in the composition that is associated with (such as in) the nanoparticles; (18) the osmolality of the composition; (19) the viscosity of the composition; (20) the pH of the composition; (21) the stability of the nanoparticles in the composition; (22) the amount of residual solvent in the composition; (23) the zeta potential of the nanoparticles in the composition; (24) the crystalline status of the rapamycin in the nanoparticles; (25) the particle morphology of the nanoparticles, such as the shape, sphericity, thickness of the coating, and/or surface-to-volume ratio; (26) the weight percentage of seco-rapamycin in the nanoparticles, as compared to the sum of seco-rapamycin and rapamycin, by weight; (27) the presence, percentage, or concentration of albumin stabilizer (such as sodium caprylate and/or N-acetyltryptophanate) in the composition; (28) the recovery of rapamycin following filtration; (29) in vitro release kinetics of the nanoparticles; (30) the portion of total rapamycin in the composition that is both in the non-nanoparticle portion of the composition and not bound to albumin; and/or (31) the weight percentage of seco-rapamycin in the composition, as compared to the sum of seco-rapamycin and rapamycin, by weight. As used herein, “albumin oligomers” or “oligomeric albumin” refers to lower molecular weight polymeric albumin species associated with a UV-absorbance-based size-exclusion chromatography peak observed between a peak associated with albumin dimers and higher molecular weight polymeric albumin species. In some embodiments, the oligomeric status (such as the percentage of albumin monomers, dimers, oligomers, or polymers (other than oligomers)) of the nanoparticles, the non-nanoparticle portion, or the total composition is assessed by size-exclusion chromatography using a mobile phase containing an aqueous portion and a miscible organic portion (such as an aqueous buffer containing 7.5% methanol) coupled with a UV detector. In some embodiments, the percentage of albumin in the nanoparticle portion that is in the form of monomeric, dimeric, oligomeric, or polymeric albumin (other than oligomeric albumin) is determined by separating the nanoparticles from the non-nanoparticle portion, dissolving the nanoparticles, and subjecting the dissolved nanoparticles to size-exclusion chromatography. In some embodiments, the size-exclusion chromatography uses a mobile phase containing an aqueous portion and a miscible organic portion (such as an aqueous buffer containing 7.5% methanol) coupled with a UV detector.

In some embodiments, the nanoparticle composition has one or more of the following distinct characteristics: (1) about 80% to about 95% (or as further provided herein) of the total albumin in the composition is in the form of monomeric albumin; (2) about 4% to about 15% (or as further provided herein) of the total albumin in the composition is in the form of dimeric albumin; (3) about 0.5% to about 5% (or as further provided herein) of the total albumin in the composition is in the form of polymeric albumin (or trimeric albumin); (4) the weight ratio of the total albumin to the total rapamycin in the composition is about 1:1 to about 10:1 (or as further provided herein); (5) about 90% or more (or as further provided herein) of the total rapamycin in the composition is in the nanoparticles; (6) about 90% or more (or as further provided herein) of the total albumin in the composition is in the non-nanoparticle portion of the nanoparticles; (7) the composition comprises tert-butanol at a concentration of less than about 10 μg/mL or less than about 10 ppm (or as further provided herein); (8) the composition comprises chloroform at a concentration of less than about 5 μg/mL or less than about 5 ppm (or as further provided herein); (9) the composition comprises an albumin stabilizer (such as sodium caprylate and/or N-acetyltryptophanate); (10) at least about 80% or more (or as further provided herein) of the rapamycin in the composition is recoverable after filtering the composition with a 0.2 micron filter; (11) the composition is stable for at least 24 hours; and/or (12) less than about 5% of the total rapamycin in the composition is both in the non-nanoparticle portion of the composition and unbound to albumin in the non-nanoparticle portion of the composition. In some embodiments, the nanoparticle composition may be a nanoparticle suspension, and the nanoparticle composition may have one or more of the following distinct characteristics (in addition to or in alternative to any one of the previously described district characteristics): (1) the concentration of albumin in the composition is about 30 mg/mL to about 100 mg/mL (or as further provided herein); (2) the concentration of rapamycin in the composition is about 1 mg/mL to about 15 mg/mL (or as further provided herein, such as about 1 mg/mL to about 7 mg/mL); (3) the osmolality of the composition is about 300 mOsm/kg to about 350 mOsm/kg (or as otherwise provided herein); (4) the viscosity of the composition is about 1.2 cP to about 1.5 cP (or as otherwise provided herein); and/or (5) the pH of the composition is about 6.0 to about 7.5 (or as otherwise provided herein).

In some embodiments, the nanoparticles of the composition have one or more of the following distinct characteristics: (1) about 70% to about 85% (or as otherwise provided herein) of the albumin in the nanoparticles is in the form of albumin monomers; (2) about 9% to about 20% (or as otherwise provided herein) of the albumin in the nanoparticles is in the form of albumin dimers; (3) about 5% to about 15% (or as otherwise provided herein) of the albumin in the nanoparticles is in the form of albumin polymers (or albumin trimers); (4) the nanoparticles have a volume weighted mean particle size and/or Z-average particle size of about 200 nm or less (or as otherwise provided herein, such as between about 50 nm and about 200 nm); (5) the nanoparticles have a polydispersity index of less than about 0.2 (or as otherwise provided herein, such as between about 0.03 and about 0.2); (6) the span of the particle size distribution ((Dv95−Dv5)/Dv50) is about 0.8 to about 1.2 (or as otherwise provided herein); (7) the nanoparticles are about 25% to about 45% albumin by weight (or as otherwise provided herein); (8) the nanoparticles are about 55% to about 75% rapamycin by weight (or as otherwise provided herein); (9) the weight ratio of albumin to rapamycin in the nanoparticles is about 1:1 to about 1:4 (or as otherwise provided herein); (10) the zeta potential of the nanoparticles in the composition is about −25 mV to about −50 mV (or as otherwise provided herein); (11) the nanoparticles have an amorphous morphology; (12) the rapamycin in the nanoparticles has an amorphous morphology; (13) the vinyl chain of the rapamycin in the nanoparticles interacts with the albumin in the nanoparticles; (14) at least a portion (such as at least 20%, or as otherwise provided herein) of the nanoparticles in the composition are non-spherical; (15) the nanoparticles comprise less than about 2.5% seco-rapamycin (or as otherwise provided herein, such as between about 0.2% and about 2.5%) compared to the sum of seco-rapamycin and rapamycin by weight; and/or (16) the composition comprises less than 3% seco-rapamycin (or as otherwise provided herein, such as between about 0.2% and about 2.5%) compared to the sum of seco-rapamycin and rapamycin by weight. In some embodiments, the nanoparticle composition may be a nanoparticle suspension, and in some embodiments the concentration of the albumin in the nanoparticle suspension that is in the nanoparticles is about 1.8 mg/mL to about 3 mg/mL (or as otherwise provided herein).

In some embodiments, the nanoparticles of the composition have one or more of the following distinct characteristics: (1) about 25% to about 50% (or as otherwise provided herein) of the albumin in the nanoparticles is in the form of albumin monomers; (2) about 5% to about 16% (or as otherwise provided herein) of the albumin in the nanoparticles is in the form of albumin dimers; (3) about 1% to about 4.5% (or as otherwise provided herein) of the albumin in the nanoparticles is in the form of albumin oligomers; (4) about 42% to about 60% (or as otherwise provided herein) of the albumin in the nanoparticles is in the form of albumin polymers (other than oligomers); (5) the nanoparticles have a volume weighted mean particle size and/or Z-average particle size of about 200 nm or less (or as otherwise provided herein, such as between about 50 nm and about 200 nm); (6) the nanoparticles have a polydispersity index of less than about 0.2 (or as otherwise provided herein, such as between about 0.03 and about 0.2); (7) the span of the particle size distribution ((Dv95−Dv5)/Dv50) is about 0.8 to about 1.2 (or as otherwise provided herein); (8) the nanoparticles are about 25% to about 45% albumin by weight (or as otherwise provided herein); (9) the nanoparticles are about 55% to about 75% rapamycin by weight (or as otherwise provided herein); (10) the weight ratio of albumin to rapamycin in the nanoparticles is about 1:1 to about 1:4 (or as otherwise provided herein); (11) the zeta potential of the nanoparticles in the composition is about −25 mV to about −50 mV (or as otherwise provided herein); (12) the nanoparticles have an amorphous morphology; (13) the rapamycin in the nanoparticles has an amorphous morphology; (14) the vinyl chain of the rapamycin in the nanoparticles interacts with the albumin in the nanoparticles; (15) at least a portion (such as at least 20%, or as otherwise provided herein) of the nanoparticles in the composition are non-spherical; (16) the nanoparticles comprise less than about 2.5% seco-rapamycin (or as otherwise provided herein, such as between about 0.2% and about 2.5%) compared to the sum of seco-rapamycin and rapamycin by weight; and/or (17) the composition comprises less than about 3% seco-rapamycin (or as otherwise provided herein, such as between about 0.2% and about 3%) compared to the sum of seco-rapamycin and rapamycin, by weight. In some embodiments, the nanoparticle composition may be a nanoparticle suspension, and in some embodiments the concentration of the albumin in the nanoparticle suspension that is in the nanoparticles is about 1.8 mg/mL to about 3 mg/mL (or as otherwise provided herein).

In some embodiments, the non-nanoparticle portion of the composition has one or more of the following distinct characteristics: (1) about 80% to about 95% (or as otherwise provided herein) of the albumin in the non-nanoparticle portion of the composition is in the form of albumin monomers; (2) about 5% to about 14% (or as otherwise provided herein) of the albumin in the non-nanoparticle portion of the composition is in the form of albumin dimers; and/or (3) about 1% to about 5% (or as otherwise provided herein) of the albumin in the non-nanoparticle portion of the composition is in the form of albumin polymers (or albumin trimers). In some embodiments, the nanoparticle composition may be a nanoparticle suspension, and the non-nanoparticle portion of the nanoparticle suspension may have one or more of the following distinct characteristics (in addition to or in alternative to any one of the previously described district characteristics): (1) the concentration of albumin in the non-nanoparticle portion of the composition is between about 30 mg/mL and about 100 mg/mL (or as otherwise provided herein); and/or (2) the concentration of rapamycin in the non-nanoparticle portion is about 20 μg/mL to about 55 μg/mL (or as otherwise provided herein).

In some embodiments, the non-nanoparticle portion of the composition has one or more of the following distinct characteristics: (1) about 80% to about 95% (or as otherwise provided herein) of the albumin in the non-nanoparticle portion of the composition is in the form of albumin monomers; (2) about 5% to about 16% (or as otherwise provided herein) of the albumin in the non-nanoparticle portion of the composition is in the form of albumin dimers; about 0.5% to about 4% (or as otherwise provided herein) of the albumin in the non-nanoparticle portion of the composition is in the form of albumin oligomers; and/or (4) about 0.5% to about 3% (or as otherwise provided herein) of the albumin in the non-nanoparticle portion of the composition is in the form of albumin polymers (other than oligomers). In some embodiments, the nanoparticle composition may be a nanoparticle suspension, and the non-nanoparticle portion of the nanoparticle suspension may have one or more of the following distinct characteristics (in addition to or in alternative to any one of the previously described district characteristics): (1) the concentration of albumin in the non-nanoparticle portion of the composition is between about 30 mg/mL and about 100 mg/mL (or as otherwise provided herein); and/or (2) the concentration of rapamycin in the non-nanoparticle portion is about 20 μg/mL to about 55 μg/mL (or as otherwise provided herein).

The compositions (such as pharmaceutical compositions) described herein can be in liquid (e.g., as a nanoparticle suspension) or powder forms. For example, in some embodiments, the composition is a liquid nanoparticle suspension (for example prior to lyophilization). In some embodiments, the composition is a reconstituted suspension (e.g., in an aqueous solution such as a saline solution). In some embodiments, the composition is dried, such as lyophilized. In some embodiments, the composition is sterile. In some embodiments, the composition is contained in a sealed container, such as a sealed vial (e.g., a glass vial) or sealed bag.

In some embodiments, the nanoparticle composition comprises (a) nanoparticles comprising rapamycin and albumin (such as human albumin), and (b) a non-nanoparticle portion comprising albumin (such as human albumin) and rapamycin. In some embodiments, about 0.5% to about 5% of the albumin in the non-nanoparticle portion or the total albumin in the nanoparticle composition is in the form of polymeric albumin (or trimeric albumin). In some embodiments, about 4% to about 14% of the albumin in the non-nanoparticle portion or the total albumin in the nanoparticle composition is in the form of dimeric albumin. In some embodiments, about 80% to about 95% of the albumin in the non-nanoparticle portion or the total albumin in the nanoparticle composition is in the form of monomeric albumin. In some embodiments, the weight ratio of the albumin to the rapamycin in the composition is about 1:1 to about 10:1. In some embodiments, about 90% or more of the albumin in the composition is in the non-nanoparticle portion. In some embodiments, about 90% or more of the rapamycin in the composition is in the nanoparticles. In some embodiments, the concentration of albumin in the nanoparticle composition that is in the non-nanoparticle portion or the concentration of total albumin in the nanoparticle composition is about 30 mg/mL to about 100 mg/mL. In some embodiments, the osmolality of the composition is about 300 mOsm/kg to about 350 mOsm/kg. In some embodiments, the viscosity of the composition is about 1.2 cP to about 1.5 cP. In some embodiments, the pH of the composition is about 6.0 to about 7.5. In some embodiments, the composition is stable at 4° C. and/or 25° C. for at least 24 hours. In some embodiments, the rapamycin in the nanoparticles has an amorphous morphology. In some embodiment, the nanoparticle composition is a nanoparticle suspension. In some embodiments, the nanoparticle composition is a dried composition. In some embodiments, the nanoparticle composition is sterile, for example by filtration. In some embodiments, the nanoparticle composition is contained within a sealed container, such as a sealed vial or a sealed bag. In some embodiments, the nanoparticle composition comprises less than 10 μg/mL tert-butanol and/or comprises less than 5 μg/mL chloroform.

In some embodiments, the nanoparticle composition comprises (a) nanoparticles comprising rapamycin and albumin (such as human albumin), wherein about 70% to about 85% of the albumin in the nanoparticles is in the form of monomeric albumin; and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 25% to about 50% of the albumin in the nanoparticles is in the form of monomeric albumin; and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 25% to about 50% of the albumin in the nanoparticles is in the form of polymeric albumin (other than oligomeric albumin); and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 9% to about 20% of the albumin in the nanoparticles is in the form of dimeric albumin; and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 5% to about 16% of the albumin in the nanoparticles is in the form of dimeric albumin; and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 25% to about 50% of the albumin in the nanoparticles is in the form of monomeric albumin, about 1% to about 4.5% of the albumin in the nanoparticles is in the form of oligomeric albumin, about 5% to about 16% of the albumin in the nanoparticles is in the form of dimeric albumin, and about 25% to about 50% of the albumin in the nanoparticles is in the form of polymeric albumin (other than oligomeric albumin); and (b) a non-nanoparticle portion comprising albumin (such as human albumin) and rapamycin.

In some embodiments, the nanoparticle composition comprises (a) nanoparticles having a Z-average particle size of about 200 nm or less (such as about 50 nm to about 200 nm), comprising rapamycin and albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 200 nm or less (such as about 50 nm to about 200 nm), comprising a coating comprising albumin (such as human albumin) and a core comprising rapamycin, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 200 nm or less (such as about 50 nm to about 200 nm), comprising about 55% to about 65% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 200 nm or less (such as about 50 nm to about 200 nm), comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 200 nm or less (such as about 50 nm to about 200 nm), comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 200 nm or less (such as about 50 nm to about 200 nm), comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 200 nm or less (such as about 50 nm to about 200 nm) and a zeta potential of about −25 mV to about −50 mV, comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 200 nm or less (such as about 50 nm to about 200 nm) and a zeta potential of about −25 mV to about −50 mV, comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 200 nm or less (such as about 50 nm to about 200 nm) and a zeta potential of about −25 mV to about −50 mV, comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein about 3% or less of the rapamycin in the nanoparticle composition is free rapamycin.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein about 3% or less of the rapamycin in the nanoparticle composition is free rapamycin.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising about 55% to about 75% (by weight) rapamycin and about 25% to about 45% (by weight) albumin (such as human albumin), wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein the sum of seco-rapamycin and rapamycin in the nanoparticles is less than 3% (such as about 0.2% to about 3%) seco-rapamycin, by weight. In some embodiments, the sum of seco-rapamycin and rapamycin in the composition is less than 3% (such as about 0.2% to about 3%) seco-rapamycin, by weight.

In some embodiments, the nanoparticle composition comprises (a) nanoparticles having a Z-average particle size of about 200 nm or less (such as about 50 nm to about 200 nm) and a zeta potential of about −25 mV to about −50 mV, comprising a coating comprising albumin (such as human albumin) and a core comprising rapamycin, wherein the albumin comprises about 25% to about 45% of the nanoparticles by weight and the rapamycin comprises about 55% to about 75% of the nanoparticles by weight, wherein about 70% to about 85% of the albumin in the nanoparticles is in the form of monomeric albumin, about 9% to about 20% of the albumin in the nanoparticles is in the form of dimeric albumin, and about 5% to about 15% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein the sum of seco-rapamycin and rapamycin in the nanoparticles is less than 3% (such as about 0.2% to about 3%) seco-rapamycin, by weight. In some embodiments, the seco-rapamycin is less than 3% (such as about 0.2% to about 3%) of the sum of seco-rapamycin and rapamycin in the composition.

In some embodiments, the nanoparticle composition comprises (a) nanoparticles comprising rapamycin and albumin (such as human albumin), wherein about 74% to about 80% of the albumin in the nanoparticles is in the form of monomeric albumin; and (b) a non-nanoparticle portion comprising albumin (such as human albumin) and rapamycin. In some embodiments, about 1.5% to about 3% of the albumin in the non-nanoparticle portion or the total albumin in the nanoparticle composition is in the form of polymeric albumin (or trimeric albumin). In some embodiments, about 7% to about 11% of the albumin in the non-nanoparticle portion in the nanoparticle composition is in the form of dimeric albumin. In some embodiments, about 7% to about 11% of the total albumin in the nanoparticle composition is in the form of dimeric albumin. In some embodiments, about 83% to about 92% of the albumin in the non-nanoparticle portion or the total albumin in the nanoparticle composition is in the form of monomeric albumin. In some embodiments, the weight ratio of the albumin to the rapamycin in the composition is about 7:1 to about 9:1. In some embodiments, about 95% or more of the albumin in the composition is in the non-nanoparticle portion. In some embodiments, about 98% to about 99.5% of the rapamycin in the composition is in the nanoparticles. In some embodiments, the concentration of albumin in the nanoparticle composition that is in the non-nanoparticle portion or the concentration of total albumin in the nanoparticle composition is about 35 mg/mL to about 45 mg/mL.

In some embodiments, the nanoparticle composition comprises (a) nanoparticles comprising rapamycin and albumin (such as human albumin), wherein about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 12% to about 17% of the albumin in the nanoparticles is in the form of dimeric albumin; and (b) a non-nanoparticle portion comprising albumin (such as human albumin) and rapamycin.

In some embodiments, the nanoparticle composition comprises (a) nanoparticles comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm, comprising rapamycin and albumin (such as human albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm, comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm, comprising a coating comprising albumin (such as human albumin) and a core comprising rapamycin, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a zeta potential of about −33 mV to about −39 mV, comprising rapamycin and albumin (such as human albumin); and (b) a non-nanoparticle portion comprising albumin (such as human albumin) and rapamycin.

In some embodiments, the nanoparticle composition comprises (a) nanoparticles having a zeta potential of about −33 mV to about −39 mV, comprising a coating comprising albumin (such as human albumin) and a core comprising rapamycin; and (b) a non-nanoparticle portion comprising albumin (such as human albumin) and rapamycin

In some embodiments, the nanoparticle composition comprises (a) nanoparticles having a zeta potential of about −33 mV to about −39 mV, comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a zeta potential of about −33 mV to about −39 mV, comprising a coating comprising albumin (such as human albumin) and a core comprising rapamycin, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm and a zeta potential of about −33 mV to about −39 mV, comprising rapamycin and albumin (such as human albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm and a zeta potential of about −33 mV to about −39 mV, comprising a coating comprising albumin (such as human albumin) and a core comprising rapamycin; and (b) a non-nanoparticle portion comprising albumin (such as human albumin) and rapamycin.

In some embodiments, the nanoparticle composition comprises (a) nanoparticles having a Z-average particle size of about 85 nm to about 95 nm and a zeta potential of about −33 mV to about −39 mV, comprising rapamycin and albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm and a zeta potential of about −33 mV to about −39 mV, comprising a coating comprising albumin (such as human albumin) and a core comprising rapamycin, wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin.

In some embodiments, the nanoparticle composition comprises (a) nanoparticles having a Z-average particle size of about 85 nm to about 95 nm, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 85 nm to about 95 nm and a zeta potential of about −33 mV to about −39 mV, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL).

In some embodiments, the nanoparticle composition comprises (a) nanoparticles having a Z-average particle size of about 85 nm to about 95 nm and a zeta potential of about −33 mV to about −39 mV, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein about 1% or less of the rapamycin in the nanoparticle composition is free rapamycin.

In some embodiments, the nanoparticle composition comprises (a) nanoparticles having a Z-average particle size of about 85 nm to about 95 nm and a zeta potential of about of about −33 mV to about −39 mV, comprising about 62% to about 68% (by weight) rapamycin and about 32% to about 38% (by weight) albumin (such as human albumin), wherein about 74% to about 80% of the albumin in the nanoparticles is in the form of monomeric albumin, about 12% to about 17% of the albumin in the nanoparticles is in the form of dimeric albumin, and about 7% to about 11% of the albumin in the nanoparticles is in the form of polymeric albumin (or trimeric albumin); and (b) a non-nanoparticle portion comprising albumin (such as human albumin) and rapamycin; wherein the concentration of the rapamycin in the nanoparticle composition is about 1 mg/mL to about 100 mg/mL (such as about 1 mg/mL to about 15 mg/mL); and wherein the sum of seco-rapamycin and rapamycin in the nanoparticles is less than 1% (such as about 0.5% to about 1%) seco-rapamycin, by weight. In some embodiments, seco-rapamycin is greater than about 0.2% (such as about 0.2% to about 3%) of the sum of seco-rapamycin and rapamycin in the composition.

Also provided herein are commercial batches of the nanoparticle compositions (such as the pharmaceutical compositions) for use of any one of the treatment methods described here. “Commercial batch” as used herein refers to a batch size that is at least about 20 grams (by mass of rapamycin). Commercial batches are produced at a larger scale than experimental or bench-scale batches. The increased scale is associated with longer production times, including longer steps (such as evaporation steps) or longer hold times between steps.

In some embodiments, the mTOR inhibitor nanoparticle composition (e.g., a sirolimus/albumin nanoparticle composition, such as FYARRO™) is administered subcutaneously. In some embodiments, the mTOR inhibitor nanoparticle composition (e.g., a sirolimus/albumin nanoparticle composition, such as FYARRO™) is administered intravenously. In some embodiments, the mTOR inhibitor nanoparticle composition (e.g., a sirolimus/albumin nanoparticle composition, such as FYARRO™) is administered at a dose between about 1 mg/m² and about 150 mg/m², between about 5 mg/m² and about 75 mg/m², e.g., via intravenous infusion. In some embodiments, the mTOR inhibitor nanoparticle composition (e.g., a sirolimus/albumin nanoparticle composition, such as FYARRO™) is administered at a dose of about any one of 5, 7.5, 10, 15, 30, 56, 75 or 100 mg/m², e.g., via intravenous infusion. In some embodiments, the mTOR inhibitor nanoparticle composition (e.g., a sirolimus/albumin nanoparticle composition, such as FYARRO™) is administered to the individual having cancer in one or more 21-day cycles (e.g., three-week cycles). In some embodiments, the mTOR inhibitor nanoparticle composition (e.g., a sirolimus/albumin nanoparticle composition, such as FYARRO™) is administered to the individual once during each 21-day cycle (e.g., three-week cycle). In some embodiments, the mTOR inhibitor nanoparticle composition (e.g., a sirolimus/albumin nanoparticle composition, such as FYARRO™) is administered during Week 1, Week 2, or Week 3 during each 21-day cycle (e.g., three-week cycle). In some embodiments, the mTOR inhibitor nanoparticle composition (e.g., a sirolimus/albumin nanoparticle composition, such as FYARRO™) is administered on Day 1, Day 8, or Day 15 of each 21-day cycle (e.g., three-week cycle). In some embodiments, the mTOR inhibitor nanoparticle composition (e.g., a sirolimus/albumin nanoparticle composition, such as FYARRO™) is administered to the individual twice during each 21-day cycle (e.g., three-week cycle). In some embodiments, the mTOR inhibitor nanoparticle composition (e.g., a sirolimus/albumin nanoparticle composition, such as FYARRO™) is administered during Week 1 and Week 2 during each 21-day cycle (e.g., three-week cycle). In some embodiments, the mTOR inhibitor nanoparticle composition (e.g., a sirolimus/albumin nanoparticle composition, such as FYARRO™) is administered during Week 2 and Week 3 during each 21-day cycle (e.g., three-week cycle). In some embodiments, the mTOR inhibitor nanoparticle composition (e.g., a sirolimus/albumin nanoparticle composition, such as FYARRO™) is administered during Week 1 and Week 3 during each 21-day cycle (e.g., three-week cycle). In some embodiments, the mTOR inhibitor nanoparticle composition (e.g., a sirolimus/albumin nanoparticle composition, such as FYARRO™) is administered on Day 1 and Day 8 of each 21-day cycle (e.g., three-week cycle). In some embodiments, the mTOR inhibitor nanoparticle composition (e.g., a sirolimus/albumin nanoparticle composition, such as FYARRO™) is administered on Day 1 and Day 15 of each 21-day cycle (e.g., three-week cycle). In some embodiments, the mTOR inhibitor nanoparticle composition (e.g., a sirolimus/albumin nanoparticle composition, such as FYARRO™) is administered on Day 8 and Day 15 of each 21-day cycle (e.g., three-week cycle). In some embodiments, the mTOR inhibitor nanoparticle composition (e.g., a sirolimus/albumin nanoparticle composition, such as FYARRO™) is administered to the individual three times during each 21-day cycle (e.g., three-week cycle). In some embodiments, the mTOR inhibitor nanoparticle composition (e.g., a sirolimus/albumin nanoparticle composition, such as FYARRO™) is administered during Week 1, Week 2, and Week 3 during each 21-day cycle (e.g., three-week cycle). In some embodiments, the mTOR inhibitor nanoparticle composition (e.g., a sirolimus/albumin nanoparticle composition, such as FYARRO™) is administered on Day 1, Day 8, and Day 15 of each 21-day cycle (e.g., three-week cycle). In some embodiments, the dosage of the mTOR inhibitor nanoparticle composition (e.g., a sirolimus/albumin nanoparticle composition, such as FYARRO™) is modified (e.g., if the individual experiences one or more adverse effects). Details regarding dosage modification for FYARRO™ and circumstances under which dosage modifications are made are detailed at www(dot)accessdata(dot)fda(dot)gov/drugsatfda_docs/label/2021/213312lbl.pdf.

### IV. Estrogen Suppressors

The methods of treatment described herein comprise administering an estrogen suppressor. As taught herein, estrogen suppressors may act as an agent that suppresses estrogen or as an agent that suppresses estrogen receptor signaling. Accordingly, one of ordinary skill in the art will readily recognize that the scope of agents encompassed by estrogen suppressors covers agents with varying mechanisms of action. For example, estrogen suppressors encompass agents that, e.g., are one or more of the following: agents that reduce (including inhibit) production of estrogen, agents that bind to estrogen, agents that reduce (including inhibit) estrogen from binding to an estrogen receptor, or agents that reduce (including inhibit) downstream events caused by estrogen binding to a receptor. The biology of human estrogen production and pathways is well known in the art, e.g., Cui et al., Trends Mol Med, 19, 2013, which is hereby incorporated herein by reference in its entirety.

The term “estrogen,” as used herein, may be used to interchangeably describe one or more of a number of estrogen compounds, including estrone, estradiol, and estriol. Estrogen suppressors, such as an agent that suppresses estrogen, may suppress any one or more of the compounds referred to herein as estrogen. In some embodiments, the estrogen suppressor suppresses estrone. In some embodiments, the estrogen suppressor suppresses estradiol. In some embodiments, the estrogen suppressor suppresses estriol.

In some embodiments, the estrogen suppressor is an agent that suppresses estrogen production. In some embodiments, the estrogen suppressor is an agent that suppresses estrogen activity, e.g., an agent that suppresses estrogen receptor signaling.

In some embodiments, the agent that suppresses estrogen production is an aromatase inhibitor. In some embodiments, the aromatase inhibitor is a type I inhibitor, which has a steroidal structure similar to androgens and inactivate aromatase by irreversibly blocking the substrate-binding site (in certain embodiments such compounds are also referred to as aromatase inactivators). In some embodiments, the aromatase inhibitor is a type II inhibitor, which are nonsteroidal and reversibly bind aromatase (e.g., letrozole). In some embodiments, the type II inhibitor aromatase inhibitor is a triazole derivative. In some embodiments, the agent that suppresses estrogen production is selected from the group consisting of letrozole, anastrozole, formestane, and exemestane. In some embodiments, the agent that suppresses estrogen production is letrozole.

In some embodiments, the estrogen suppressor is an agent that suppresses estrogen activity, such as suppresses estrogen receptor signaling. In some embodiments, the agent that suppresses estrogen activity is an estrogen receptor antagonist. One of ordinary skill in the art will readily appreciate that there are a number of forms of estrogen receptor antagonists that have varying associated mechanisms of action. For example, in some embodiments, the estrogen receptor antagonist is a pure antiestrogen, which blocks estrogen function. In some embodiments, the estrogen receptor antagonist is a selective estrogen receptor downregulator/degrader (SERD), e.g., fulvestrant. In some embodiments, the estrogen receptor antagonist is a selective estrogen receptor modulator (SERM), which selectively modulates estrogen receptors typically by tissue type. In some embodiments, the estrogen receptor antagonist is a dual-mechanism estrogen receptor inhibitor. In some embodiments, the estrogen receptor antagonist is a competitive inhibitor of estrogen receptor binding. In some embodiments, the estrogen receptor antagonist is an estradiol analogue, e.g., fulvestrant. In some embodiments, the agent that suppresses estrogen activity is selected from the group consisting of fulvestrant, elacestrant, tamoxifen, hydroxyprogesterone caprate, droloxifene, ormeloxifene, toremifene, faloxifene, raloxifene, and clomiphene. In some embodiments, the agent that suppresses estrogen activity is fulvestrant.

In some embodiments, the estrogen receptor antagonist is not a SERM.

Exemplary dosages, routes of administration, and dosing schedules for an estrogen suppressor are provided in other sections herein. In an effort to exemplify the teachings provided herein, certain additional embodiments regarding estrogen receptor antagonists are provided below. For example, in some embodiments, the estrogen suppressor is letrozole. In some embodiments, the estrogen suppressor is letrozole, wherein letrozole is administered to the individual orally. In some embodiments, letrozole is administered to the individual at an amount of about 0.1 mg to about 10 mg, such as about any of 0.2 mg, 0.3 mg, 0.4 mg, 0.5 mg, 0.6 mg, 0.7 mg, 0.8 mg, 0.9 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg. In some embodiments, letrozole is administered to the individual daily.

In some embodiments, the estrogen suppressor is fulvestrant. In some embodiments, the estrogen suppressor is fulvestrant, wherein fulvestrant is administered to the individual intramuscularly. In some embodiments, fulvestrant is administered to the individual at an amount of about 100 mg to about 700 mg, such as about any of 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, or 700 mg. In some embodiments, fulvestrant is administered to the individual using an initiation cycle and a maintenance cycle. In some embodiments, fulvestrant is administered to the individual on days 1, 15, and 29 of an initiation cycle followed by one or more monthly maintenance cycle.

### V. Articles of Manufacture and Kits

In some embodiments, there is provided an article of manufacture containing materials useful for the treatment of a hormone-dependent cancer (including an endometrial cancer (e.g., endometrioid endometrial cancer) or a hormone receptor positive breast cancer), the article of manufacture, such as a medicament or medicament combination, comprising an mTOR inhibitor nanoparticle composition (such as sirolimus/albumin nanoparticle composition, e.g., nab-sirolimus) and an estrogen suppressor (e.g., letrozole or fulvestrant). The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is (a) a nanoparticle formulation of an mTOR inhibitor (e.g., nab-sirolimus); or (b) an estrogen suppressor (e.g., letrozole or fulvestrant). The label or package insert indicates that the composition is used for treating the particular condition in an individual, such as described herein. The label or package insert will further comprise instructions for administering the composition to the individual according to the methods described herein. Articles of manufacture and kits comprising combination therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a hormone-dependent cancer (e.g., an endometrial cancer (e.g., endometrioid endometrial cancer) or hormone-receptor positive breast cancer).

Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a hormone-dependent cancer (e.g., an endometrial cancer (e.g., endometrioid endometrial cancer) or hormone-receptor positive breast cancer). Kits of the invention include one or more containers comprising an mTOR inhibitor nanoparticle composition (such as sirolimus/albumin nanoparticle composition) (or unit dosage form and/or article of manufacture), and in some embodiments, further comprise an estrogen suppressor (e.g., letrozole or fulvestrant) and/or instructions for use in accordance with any of the methods described herein. The kit may further comprise a description of selection of individuals suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the mTOR inhibitor nanoparticle composition (such as sirolimus/albumin nanoparticle composition) and an estrogen suppressor (e.g., letrozole or fulvestrant) generally include information as to dosage, dosing schedule, and route of administration for the intended treatment. The containers may be unit doses, bulk packages (e.g., multi-dose packages) or sub-unit doses. For example, kits may be provided that contain sufficient dosages of an mTOR inhibitor nanoparticle composition (such as sirolimus/albumin nanoparticle composition) and an estrogen suppressor (e.g., letrozole or fulvestrant) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the mTOR inhibitor nanoparticle composition (such as sirolimus/albumin nanoparticle composition) and an estrogen suppressor (e.g., letrozole or fulvestrant) and instructions for use, packaged in quantities sufficient for storage and use in pharmacies, for example, hospital pharmacies and compounding pharmacies.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example 1

This example demonstrates a study using nab-sirolimus in combination with fulvestrant (a selective estrogen receptor degrader; SERD) to treat hormone-receptor positive breast cancer cell lines.

A series of single-agent treatments of fulvestrant or nab-sirolimus and combination treatments of both fulvestrant and nab-sirolimus were separately applied to aliquots of hormone receptor positive breast cancer cell lines (either MCF7 or MDA-MB-361). The series including varying concentration of nab-sirolimus (20 nM or 80 nM) and fulvestrant (0.98 nM to 1000 nM). Following the application of the individual treatments, the cell aliquots were incubated for 5 days. Subsequently, the antiproliferative and cytotoxic effects of single agent and combination treatments were assessed using an automated trypan blue exclusion assay.

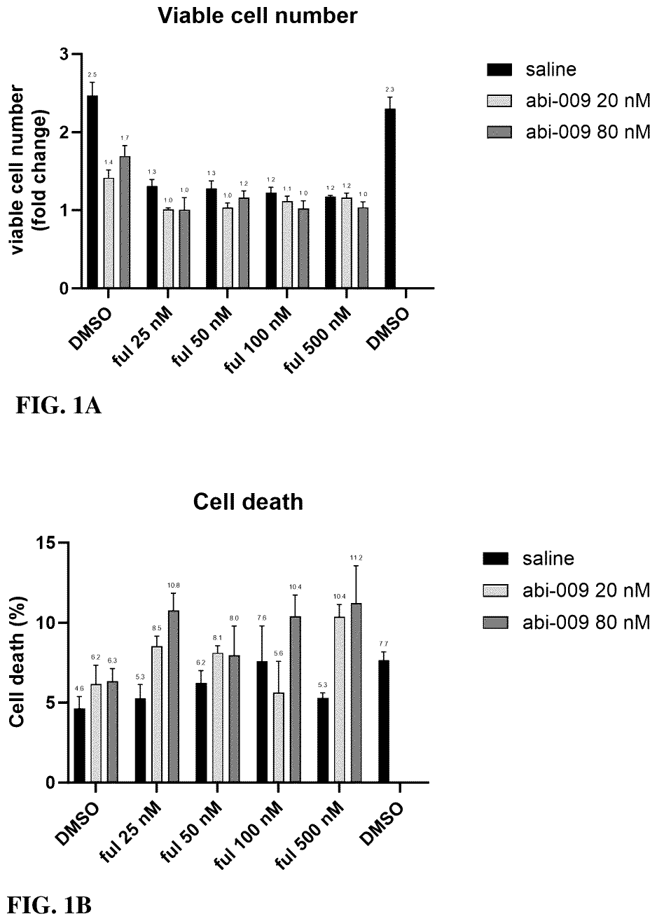

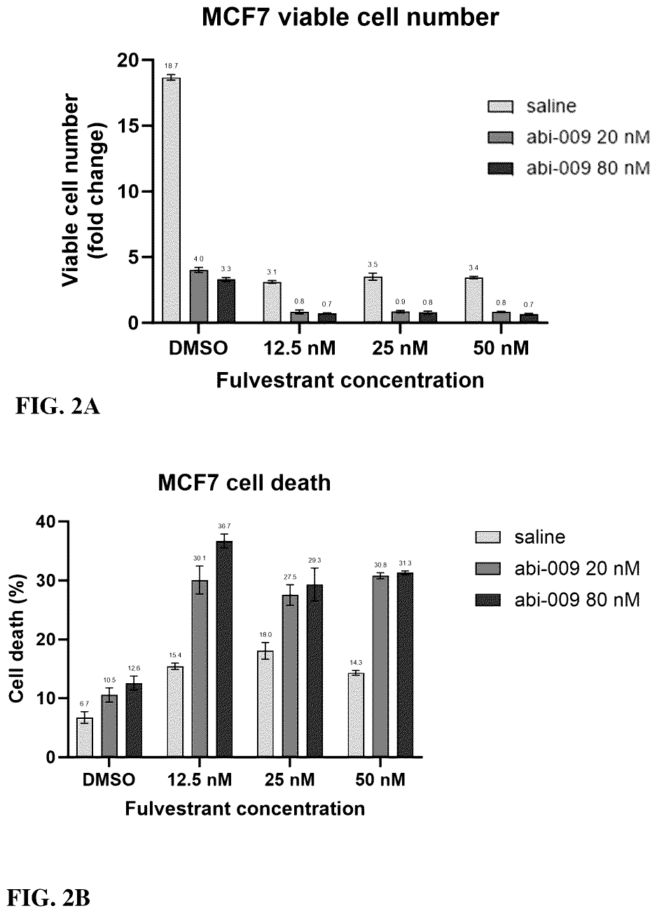

From the study, it was found that nab-sirolimus potentiated the cytotoxicity of fulvestrant. The addition of 20 nM and 80 nM nab-sirolimus to all fulvestrant doses tested markedly decreased cell viability and nearly doubled cell death in MCF7 cells as well as in MDA-MB-361 cells (FIGS. 1A and 1B for MDA-MB-361 cells: “ful” is fulvestrant; abi-009 is nab-sirolimus; bars are arranged in repeating saline, abi-009 20 nM, and abi-009 80 nM order except for the right-most DMSO data point which is a saline treatment. FIGS. 2A and 2B for MCF7 cells: abi-009 is nab-sirolimus; bars are arranged in repeating saline, abi-009 20 nM, and abi-009 80 nM order).

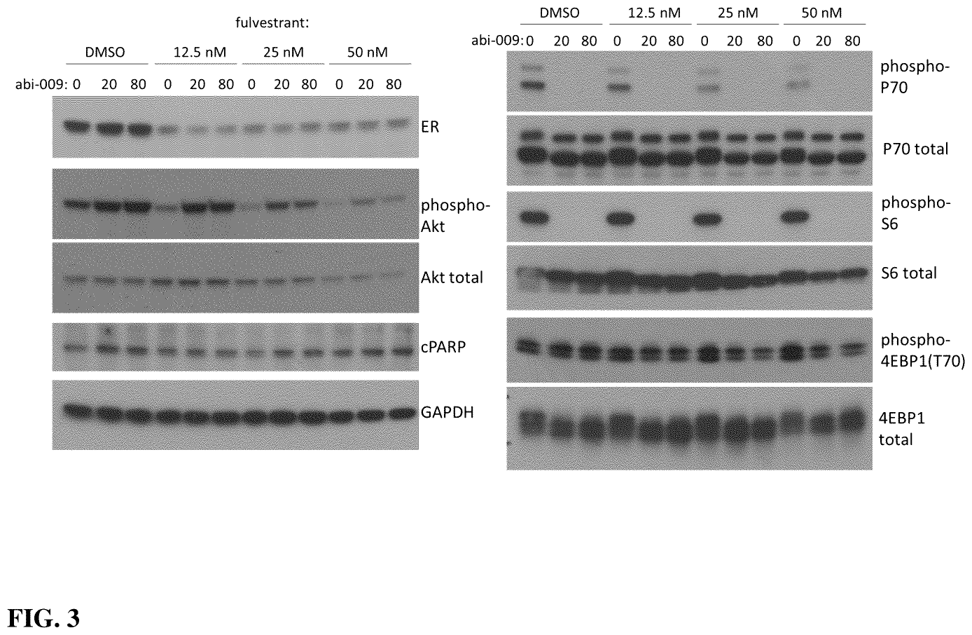

Western blot analysis was performed to assess p4EBP1 in MCF7 cells. Western blots (FIG. 3) showed increased p4EBP1 in response to fulvestrant in a dose-dependent manner, which was reversed by combination with nab-sirolimus. Thus, addition of nab-sirolimus to endocrine-based therapies, such as fulvestrant, may overcome mechanisms of resistance.

Western blot analysis was performed to assess pAKT in MCF7 cells. Western blots (FIG. 3) showed increased pAKT in response to nab-sirolimus, which was reversed by combination with fulvestrant in a dose-dependent manner. Thus, addition of fulvestrant, to mTOR targeting agents, such as nab-sirolimus may overcome mechanisms of resistance.

### Example 2

A Phase 2 Multi-Center Open-Label Trial of Nab-Sirolimus in Combination with Letrozole in Advanced or Recurrent Endometrioid Endometrial Cancer

A prospective Phase 2, open-label, multi-institutional study is designed to evaluate the efficacy and safety of nab-sirolimus+letrozole in patients with advanced (unresectable International Federation of Gynecology and Obstetrics [FIGO] Stage III or IV) or recurrent endometrioid endometrial carcinoma (EEC) Grade ≤3 who have received both a platinum agent and a checkpoint inhibitor, unless the agent(s) are contraindicated or result in intolerance. Patients who have received prior therapy must have achieved a response to at least one prior therapy. Patients with advanced or recurrent EEC are treated with nab-sirolimus (100 mg/m² given on Days 1 and 8 in a 21-day cycle, combined with letrozole (2.5 mg orally, daily). Eligible patients are treated until unacceptable toxicity or disease progression, or until in the opinion of the Investigator the patient is no longer benefiting from therapy, or at patient discretion.

A cycle consists of 21 days. Patients receive nab-sirolimus by intravenous (IV) infusion over 30 (+10) minutes (ie, 30-40 minute infusion) on Days 1 and 8 of every 21-day cycle (2/3 weekly schedule) at a dose of 100 mg/m². Letrozole is administered at a dose of 2.5 mg orally daily of every 21-day cycle. Up to 4 sequential dose reductions of nab-sirolimus are permitted (75, 56, 45, and 35 mg/m²) for management of adverse events (AEs). If among the first treated patients, 3/6 or 4/10 require a nab-sirolimus dose reduction within the first 4 months, the starting dose for all subsequently enrolled patients is 75 mg/m² nab-sirolimus (with no subsequent dose escalation permitted).

Approximately 29 patients at approximately 8 sites in the US will be enrolled in the study. The study duration is approximately 24 months from first patient enrolled to last patient follow-up, including approximately 18 months of enrollment period (from first to last patient in), allowing a minimum of 6 months of treatment for all enrolled patients, a 28-day screening and a 28-day (4 week) safety follow-up after the last dose.

End of Treatment (EOT) for a patient is defined as the date of the last dose of nab-sirolimus. The End of Treatment visit (EOT visit) for a patient is a safety follow-up visit; safety assessments and procedures are performed 28-35 days after the last dose of nab-sirolimus is administered. End of Study (EOS) for a patient is defined as the date of last follow-up. The EOS is defined as either the date of the last visit of the last patient to complete the study, or the date of receipt of the last data point from the last patient that is required for primary, secondary, and/or exploratory analysis, as pre-specified in the protocol.

The Follow-up period begins after the EOT visit. All patients that discontinue the study treatment and have not withdrawn full consent to participate in the study participate in the follow-up phase for survival and initiation of new therapy, including surgery or anticancer therapy. If, after nab-sirolimus treatment, a patient is able to undergo surgical resection of her primary tumor with curative intent, documentation on outcome and disease-free or stable disease status is followed and documented. Follow-up continues for approximately every 12 (±3) weeks, until death, withdrawal of consent, or the study closes, whichever is the earliest. This evaluation may be made by record review and/or telephone contact.

The primary endpoint is to determine the ORR (complete or partial response) in patients with advanced or recurrent EEC, receiving combination treatment of nab-sirolimus and letrozole. Based on results in studies with an mTOR inhibitor and letrozole in patients with prior lines of therapy, the historical benchmark is an ORR of 5%; to be considered a signal for meaningful clinical benefit, a 20% ORR is the target. Simon's optimal 2-stage design is used to test a clinically meaningful difference from the historical estimate of 5% ORR to 20% ORR in this study, with a type 1 error rate of 0.05, and statistical power of 80% requires a combined sample size of 29 efficacy evaluable patients (10 in the first stage and an additional 19 in the second stage). Interim analysis (Simon's stage 1) is performed after 10 efficacy evaluable patients had the opportunity to be treated for 6 months and had at least 1 post-baseline scan. If 0/10 patients experience a response, the treatment is deemed ineffective. If 1 or more patients achieve a response, an additional 19 efficacy evaluable patients may be enrolled for a total of 29 patients. The null hypothesis is rejected if 4 or more responses are observed in 29 patients.

Secondary objectives are to evaluate the duration of response (DOR), disease control rate (DCR), time to response (TTR), and progression-free survival (PFS), and overall survival (OS) of nab-sirolimus in combination with letrozole in EEC; and to establish the safety profile of nab-sirolimus in combination with letrozole in EEC. Secondary endpoints are a) DOR: Determined for patients with BOR of confirmed CR or PR; b) DCR: BOR of confirmed CR or PR (either of any duration) or stable disease (SD) 2:12 weeks following study treatment initiation; c) TTR: Time from study treatment initiation to initial measurement of CR or PR, where CR or PR is subsequently confirmed; d) PFS: number of months from study treatment initiation to the date of disease progression or death due to any cause or last tumor assessment if censored; e) OS: number of months from study treatment initiation to the date of death due to any cause or last follow up date if alive; f) incidence and severity of treatment-emergent and treatment-related adverse events (AEs) as assessed by the National Cancer Institute Common Terminology Criteria for Adverse Events (NCI CTCAE) v5.0.

The exploratory objective is to evaluate baseline molecular biomarkers and genomics and the association between molecular/genomic profile and clinical outcome. Exploratory endpoints are: a) from baseline tumor tissue (archival or fresh biopsy) and blood (peripheral blood mononuclear cells [PBMCs]): (1) to evaluate the expression of pS6, and other relevant markers via immunohistochemistry; (2) to understand the estrogen (ER) and progesterone receptor (PgR) expression status, tumor mutational burden (TMB), copy number alterations (CNAs), and microsatellite instability (MSI) status, and polymerase epsilon (POLE) mutation status; (3) to understand the mutational profile via next-generation sequencing and the association between mutational profile and clinical outcome; b) circulating tumor DNA (ctDNA) at baseline and changes from baseline during treatment.

Efficacy is assessed by Investigator radiologic review using CT or MRI scans using RECIST v1.1. Patients are evaluated for CR, PR, SD, or PD by CT imaging or MRI. The same modality of imaging is used throughout the study. Baseline scan results are accepted from outside institutions but must be performed within 4 weeks of starting therapy and must include (as clinically indicated) chest, abdominal, and pelvic (CAP) CT or MRI. The first response assessment by CT or MRI scans documenting target lesions are performed 6 weeks (±7 days) after first treatment and repeated every 6 weeks (±7 days) for the first year, then every 12 weeks (±7 days) thereafter until disease progression. If an initial observation of objective response (CR or PR) is made, a confirmation scan is performed 6 (±1) weeks after the initial observation. Scans continue on schedule regardless of delays in nab-sirolimus dosing. The BOR and DCR are reported along with exact 95% CIs computed by the Clopper-Pearson method. For PFS, OS, and DOR, the KM estimates and corresponding two-sided 95% CIs for the median and quartiles are provided. The KM plot may also be provided.

Safety and tolerability are monitored through continuous reporting of treatment-emergent and treatment-related AEs, AEs of special interest (AESIs), laboratory abnormalities, and incidence of patients experiencing dose modifications, dose delay/missed, dose interruptions, and/or premature discontinuation of IP due to an AE. All AEs are recorded by the Investigator from the time the patient signs informed consent until 28 days after the last dose of IP. Adverse events are graded by NCI CTCAE v5.0. Physical examination, vital signs, laboratory assessments (eg, serum chemistry, hematology, lipid panel, thyroid function), and performance status are monitored. All serious AEs (SAEs; regardless of relationship to IP) are followed until resolution. Local laboratory analyses are performed as per study schedule. Safety (incidence and severity of AEs and significant laboratory abnormalities) is a secondary endpoint and is analyzed using the Full Analysis Set. Patient incidence of all treatment-emergent AEs are tabulated by System Organ Class and Preferred Term. Tables of fatal adverse events, serious adverse events, treatment-related AEs, Grade 3 or 4 AEs, adverse events of special interest, and adverse events leading to withdrawal from investigation product, AEs leading to death are provided. The Medical Dictionary for Regulatory Activities (MedDRA) is used to code adverse events and the NCI CTCAE version 5.0 is used to grade severity of adverse events and laboratory toxicities. For nab-sirolimus exposure, summary statistics are provided but not limited to total number of doses, average dose administered, and duration of treatment. For select laboratory parameters, changes of laboratory values over time (eg, change from baseline summary statistics), grade shifts in laboratory values from baseline to worst on-study value, and Grade 3 or higher laboratory toxicities are summarized.

Eligible individuals must meet all of the following inclusion criteria: a) have clinically confirmed advanced (FIGO Stage III or IV) or recurrent endometroid endometrial carcinoma (EEC) (histologic documentation of the recurrence is suggested but not required); b) have 1 or more measurable target lesion at baseline by computed tomography (CT; or magnetic resonance imaging [MRI] if CT scans are contraindicated) as defined by RECIST version 1.1; c) have EEC that is metastatic or locally advanced where surgical resection is not an option or likely to result in severe morbidity; d) chemotherapy, hormonal therapy, checkpoint inhibitors, and/or other therapy is permitted, if adjuvant treatment ended ≥6 months prior to start of study therapy; e) be age: 18 years or older; f) have Eastern Cooperative Oncology Group (ECOG) performance status of 0 or 1; g) have adequate liver function as defined by: (1) Total bilirubin ≤1.5× upper limit of normal (ULN) (unless due to Gilbert s syndrome, then ≤3×ULN); (2) Aspartate aminotransferase (AST):S2.5×ULN (:S5×ULN if attributable to liver metastases); h) Adequate renal function: creatinine clearance (CrCL) 2:30 mL/min, Cockcroft-Gault:

$$CrCL = \frac{( 140 - age ) \times ( weight in kg )}{72 \times ( serum creatinine in mg / dL )} \times ( 0.85 if female ) ;$$

i) have adequate hematologic parameters as defined by: 1) Absolute neutrophil count (ANC) ≥1.0×109/L (growth factor support allowed); 2) Platelet count ≥100,000/mm3 (100×109/L) (transfusion and/or growth factor support allowed); 3) Hemoglobin ≥8.0 g/dL (transfusion and/or growth factor support allowed); i) have fasting serum triglyceride of ≤300 mg/dL; have fasting serum cholesterol of ≤350 mg/dL; j) be minimum of 4 weeks since any major surgery, completion of radiation, or completion of prior systemic anticancer therapy, or at least 5 half-lives if the prior therapy is a single agent small-molecule therapeutic, and adequately recovered from the acute toxicities of any prior therapy, including neuropathy, to Grade ≤1; k) be non-pregnant and non-breastfeeding female: Females of childbearing potential must agree to use effective contraception or abstinence without interruption from 28 days prior to starting investigational product (IP) through 3 months after the last dose of IP and have a negative serum pregnancy test (beta human chorionic gonadotropin [β-hCG]) result at screening and agree to ongoing pregnancy testing during the course of the study, and after the end of study treatment; a second form of birth control is required even if she has had a tubal ligation; l) understand and sign the informed consent; m) be able to comply with scheduled visits, laboratory tests, and other study procedures; and n) prior therapies allowed include:
a) Adjuvant setting—treatment with chemotherapy, hormonal therapy, checkpoint inhibitors, and/or other therapy is permitted as long as the adjuvant therapy ended ≥6 months from enrollment.
b) Recurrent/advanced/metastatic setting—treatment with 0-1 prior chemotherapy regimens is permitted (patients may be naïve to chemotherapy); chemotherapy must have been completed ≥3 months prior to enrollment. Patients are permitted to have received adjuvant chemotherapy and no more than 1 line of chemotherapy in the recurrent/advanced/metastatic setting.
c) Non-chemotherapy-based treatment (eg, checkpoint inhibitors, hormonal therapy, and/or small molecule agents) is permitted at any point as long as therapy ended ≥4 weeks prior to enrollment.
d) Patients who have received prior therapy in the recurrent/advanced/metastatic setting must have achieved a complete or partial response (investigator-assessed) to at least 1 therapy.

Exclusion criteria are: a) prior treatment with an mTOR inhibitor, including nab-sirolimus; b) patients with known inactivating TSC1 or TSC2 alterations (based on tissue or liquid next generation sequencing [NGS]) unless the PRECISION 1 study (NCT05103358) has been closed to enrollment; c) severe (Grade ≥3) ongoing infection requiring parenteral or oral anti-infective treatment, either ongoing or completed ≤7 days prior to enrollment; d) patients with primary refractory disease (ie, those who have never achieved a complete or partial response to prior therapy) are not permitted on study; e) patients with the following are excluded: (1) known or suspected brain metastases; (2) severe heart disease defined as unstable angina pectoris, New York Heart Association (NYHA) Class III or IV congestive heart failure, myocardial infarction ≤6 months prior to first study treatment, serious uncontrolled cardiac arrhythmia or any other clinically significant cardiac disease; (3) severe lung disease defined as a diffusing capacity for carbon monoxide (DLCO) that is ≤50% of normal predicted value and/or an O2 saturation ≤88% at rest on room air (Note: spirometry and pulmonary function tests [PFTs] are not required to be performed unless clinically indicated); (4) nonmalignant medical illnesses that are uncontrolled or whose control may be jeopardized by the treatment with the study therapy; (5) a history of malignancies other than the one under treatment unless the patient is disease-free for more than 5 years from completion of therapy administered with curative intent. Controlled non-melanoma skin cancers, carcinoma in situ of the cervix, resected incidental prostate cancer, certain low grade hematologic malignancies (eg, chronic lymphocytic leukemia [CLL], follicular lymphoma, etc), or other adequately treated carcinoma in situ may be eligible, after discussion with the Medical Monitor; (6) uncontrolled hypertension (systolic blood pressure ≥160 mmHg and/or diastolic blood pressure ≥100 mmHg); (7) patients with history of interstitial lung disease and/or pneumonitis, or pulmonary hypertension; (8) individuals with known human immunodeficiency virus (HIV) infection are excluded from this study as combination antiretroviral therapy could potentially result in significant pharmacokinetic interactions. In addition, these individuals are at increased risk of serious infections due to the immunosuppressive effects of mTOR inhibition; (9) active hepatitis B or hepatitis C, with detectable viral load; f) required use of concomitant medications with strong CYP3A4 interactions (induction or inhibition) should be discontinued (strong inhibitors include ketoconazole, itraconazole, voriconazole, erythromycin, clarithromycin, telithromycin; strong inducers include rifampin and rifabutin); these agents must be discontinued prior to first dose of nab-sirolimus.

The following assessments and actions are performed on Day 1 of each cycle prior to nab-sirolimus administration, unless otherwise specified: physical examination as per standard of care; vital signs (temperature, systolic and diastolic blood pressure, and heart rate); weight assessment; body surface area (BSA) calculation based on actual weight (calculated ONLY on Cycle 1/Day 1 per institutional standard methods; to be recalculated only if the weight changes by >10% in subsequent cycles); ECG (Cycle 1 and Cycle 3 only); concomitant medication and procedures evaluation; ECOG performance status assessment; baseline blood for biomarkers prior to infusion on Cycle 1 Day 1 (from enrolled patients only), then every 12 weeks thereafter, for up to 1 year; biomarker pre-treatment tissues (archival or fresh, Cycle 1 only, and from enrolled patients only); clinical chemistry panel; CBC, differential, and platelet count; thyroid function as defined by TSH (and T3 and T4, if available); fasting lipids (triglycerides, total cholesterol, HDL, and LDL cholesterol) on even numbered cycles, starting with Cycle 2; adverse event assessment; nab-Sirolimus dosing; start daily letrozole dosing. Day 1 evaluations for Cycle 1 may be omitted if screening evaluations are performed within 3 days of Cycle 1 Day 1. Laboratory assessments include chemistry, hematology, pregnancy test (women of childbearing potential, includes tubal ligation). See Table 1 for analytes tested for the required laboratory assessments on Day 1.

The following assessments and actions are performed on Day 8 of each cycle prior to nab-sirolimus administration, unless otherwise specified: vital signs (temperature, systolic and diastolic blood pressure, and heart rate); concomitant medication and procedures evaluation; adverse event assessment; nab-Sirolimus dosing; CBC, differential, and platelet count (see Table 1 for analytes to be included).

TABLE 1
Analyte Listing

| Chemistry | Hematology | Other Labs |
|---|---|---|
| Sodium | WBC | Pregnancy test |
| Potassium | RBC | Sirolimus PK (central lab) |
| Bicarbonate | Hemoglobin | Cancer antigen 125 (CA-125) |
| Chloride | Hematocrit | TSH (if indicated T3, T4) |
| Total protein | Platelets | HIV |
| Albumin | Differential: | HBV sAg |
| Calcium | Neutrophils | HBV cAb |
| Magnesium | Lymphocytes | HCV Ab |
| Phosphorus | Monocytes | Total Cholesterol |
| Glucose | Eosinophils | HDL |
| BUN | Basophils | LDL |
| Creatinine | | Triglyceride |
| Total bilirubin | | |
| Alkaline phosphatase | | |
| AST (SGOT) | | |
| ALT (SGPT) | | |
| Amylase | | |
| Lipase | | |

Patient visits are complete after the EOT visit. The EOT visit is a safety follow-up visit that is to be performed at least 4 weeks (+7 days) after the last dose of nab-sirolimus. All efforts should be made to conduct this visit. If it is not possible to conduct the EOT visit, documentation of efforts to complete the visit should be provided.

The following procedures are completed at the EOT visit as designated in the Schedule of Assessments (Table 2): Physical examination; vital signs (temperature, systolic and diastolic blood pressure, and heart rate); weight; concomitant medication and procedures evaluation; ECOG performance assessment; adverse event assessment; laboratory assessments: chemistry, CBC, differential, platelet count, pregnancy test (women of childbearing potential) (see Table 2 for analytes to be included); tissue sample for biomarker analysis (optional, banked); blood sample for biomarker analysis (banked); imaging assessment: CT/MRI is to be performed at the end of study visit only for those patients that discontinue treatment for a reason other than disease progression per RECIST v1.1

TABLE 2
Schedule of Assessments

| | | Cycle 1 through Last | | End of | |
|---|---|---|---|---|---|
| | Screening ^{b} | Cycle (Treatment | | Treatment | Follow- |
| | (Day −28 | Period) | | (EOT) | up |
| Assessment ^{a} | to −1) | Day 1 ^{c} | Day 8 | Visit ^{d} | Period ^{e} |

General Assessments

| Informed Consent | X | | | | |
|---|---|---|---|---|---|
| Demographics | X | | | | |
| Medical History | X | | | | |
| Physical Examination | X | X | | X | |
| Vital Signs, Height, and Weight ^{f} | X | X ^{f} | X ^{f} | X ^{f} | |
| BSA Calculation ^{g} | | X ^{g} | | | |
| ECG (12-lead) ^{h} | X | X ^{h} | | | |
| Prior/Concomitant | X | X | X | Until 28 days | |
| Medication/Procedures Evaluation ⁱ | | | | after the last | |
| | | | | dose of IP | |
| Performance Status (ECOG) | X | X | | X | |
| Pregnancy Test ^{j} | X | | | X | |
| Biomarker: Pre-treatment tissues | | X | | X | |
| (archival or fresh) ^{k} | | | | | |
| Biomarker: blood sample ^{k} | | X | | X | |
| Survival status | | | | | X |
| Adverse Events | Continuous starting from signing of Informed | | | | |
| | Consent until 28 days after last dose of IP ¹ | | | | |
| nab-Sirolimus Dosing | | X | X | | |

| | |
|---|---|
| Letrozole 2.5 mg orally | daily, Days 1 through 21 |

Local Laboratory Assessments (refer to Table 2)

| | | | | |
|---|---|---|---|---|
| Clinical Chemistry Panel and CA- | X | X | | X |
| 125 | | | | |
| CBC, Differential, Platelet Count | X | X | X | X |
| Thyroid function | X | X | | |
| HIV Ab, HBV sAg, HBV cAb, | X | | | |
| HCV Ab | | | | |
| Fasting Lipids ^{m} | X | m | | |

Imaging and Efficacy Assessments

| | | | |
|---|---|---|---|
| CT/MRI ⁿ | X | Every 6 weeks (±7 days) | X |
| | | after CID1 for the first | |
| | | year, then every 12 weeks | |
| | | (±7 days) thereafter | |

Abbreviations:
Ab = antibody;
AE = adverse event;
B-hCG = beta human chorionic gonadotropin;
BSA = body surface area;
C = cycle;
cAb = core antibody;
CBC = complete blood count;
CR = complete response;
CT = computed tomography;
ctDNA = circulating tumor deoxyribonucleic acid;
D = day;
ECG = electrocardiogram;
ECOG = Eastern Cooperative Oncology Group;
EOT = End of Treatment;
HBV = hepatitis B virus;
HCV = hepatitis C virus;
HIV = human immunodeficiency virus;
IP = investigational product;
MRI = magnetic resonance imaging;
PK = pharmacokinetic;
PR = partial response;
sAg = surface antigen.

Tumor response is assessed by CT or MRI scan of the chest, abdomen, and pelvis (with or without contrast as per institutional standard methods); image preparation and evaluation follow the specifications provided in the RECIST v1.1. The same modality (CT or MRI) is used at screening and throughout the study. Blood samples for biomarker analysis is collected at baseline, every 12 (±1) weeks associated with scans for the first year, and at the EOT visit/disease progression. CT/MRI scans are performed at the following frequency: ≤28 days prior to C1D1 (screening); followed by every 6 weeks (±7 days) after C1D1 for the first year; followed by every 12 weeks (±7 days) thereafter until disease progression. End of Treatment visit CT/MRI is performed only for those patients that discontinue treatment for a reason other than disease progression. An unscheduled scan for suspected disease progression can be performed at any time. However, adherence to the planned imaging schedule is critical regardless of dose delays or unscheduled or missed assessments. Determination of disease progression for clinical management of patients on study is assessed at the local site. If an initial observation of objective response (CR or PR) is made, a confirmation scan is performed 6 (±1) weeks after initial observation. At the time of disease progression, if a biopsy is performed, a tumor sample should be collected, if available. The surgical pathology report is submitted with the tumor tissue sample.

Post-treatment survival time and any subsequent initiation of anticancer therapy is collected approximately every 12 (±3) weeks from EOT visit or more frequently as needed, until death, withdrawal of consent, or the study closes, whichever is earliest. If, after nab-sirolimus treatment, a patient is able to undergo surgical resection of his/her primary tumor with curative intent, documentation on outcome and disease-free or stable disease status is followed and documented. This evaluation may be by record review and/or telephone contact.

Baseline tissue (archival or fresh biopsy) of up to 20 slides, if available, and blood samples is required from all patients. The key objectives of the baseline tissue profiling for the exploratory biomarker analysis and baseline blood biomarker analysis (PBMCs) are: to evaluate the expression of pS6, and other relevant markers via immunohistochemistry; to understand the ER and PgR expression status, TMB, CNA, and MSI status, and POLE mutation status; to understand the mutational profile via next-generation sequencing and the association between mutational profile and clinical outcome; to assess ctDNA at baseline and changes from baseline during treatment. The key objective of the longitudinal blood biomarker analysis during treatment is to evaluate genomic changes to treatment. Blood samples are collected at baseline on Cycle 1 Day 1 (prior to infusion), at every 12 (±1) week scan for the first year, and at the EOT visit or disease progression per RECIST v1.1.

Prior to nab-sirolimus administration on Days 1 and 8, patients must meet the following hematological requirements prior to dosing: ANC ≥1.0×10⁹/L, platelet count ≥100×10⁹/L, and hemoglobin ≥8.0 g/dL. If treatment cannot be administered on the planned visit date, nab-sirolimus may be administered ±3 days from the scheduled date; however, no dose is administered ≤4 days from the previous dose. The dose may be delayed for up to 21 days from the expected administration date for the dose, if needed. Approval from the Medical Monitor is required to restart study treatment after >21 days of interruption (counting from the original planned administration date).

If the dose is not administered on the planned Day 1±3 days, the cycle starts when the first dose of the cycle is actually administered. If the Day 8 dose is not administered on the planned day ±3 days, then that week becomes a week of rest. The next dose (if counts and chemistries permit) becomes Day 1 of a new cycle (ie, if Day 8 cannot be administered by Day 11 of any cycle, then the Day 8 dose of the cycle is skipped, and Day 1 of the next cycle should be planned to start 14 days after the previous dose).

Doses are reduced for hematologic and non-hematologic toxicities, as clinically indicated and as shown in Table 3. Up to 4 sequential dose reductions are permitted (75, 56, 45, and 35 mg/m²) for management of adverse events. If the patient is experiencing more than one toxicity simultaneously, then refer to the guidelines below. Dose modifications/discontinuations (refer to general guidelines and Table 4 below) are to be made according to the highest grade toxicity, or when the toxicity grades are the same, follow the guideline that is most conservative. Toxicities/AEs are graded using the NCI CTCAE v5.0.

Guidelines for dose modifications and interruptions for management of common toxicities associated with the study treatment(s) are provided below. General guidelines: In the event of any clinically significant AE(s), treatment may be withheld, and supportive therapy administered as clinically indicated. If the toxicity or event is not Grade 3 or 4 and resolves to baseline or Grade 1 within 21 days of withholding therapy, then treatment may be restarted.

Dose reduction of nab-sirolimus may be implemented for any toxicities considered intolerable of any grade, at the discretion of the Investigator. If two dose level reductions are felt to be the only safe way to resume treatment with the IP, then the Investigator should discuss with the Medical Monitor before continuing treatment with the IP. If the toxicity does not resolve to Grade 1 or baseline within 21 days, withdrawal from treatment with the IP is recommended. However, if the Investigator, patient, and Aadi Medical Monitor agree that further treatment would benefit the patient, treatment can continue with a dose level reduction.

TABLE 3
nab-Sirolimus Dose Level Reduction Guidelines

| Dose level | nab-sirolimus Dose/Schedule |
|---|---|
| 0 (initial dose) | 100 mg/m² (actual dose capped at 200 mg [BSA 2 m²]) |
| −1 (first dose reduction) | 75 mg/m²: 25% reduction of initial dose of 100 mg/m² |
| −2 (second dose reduction) | 56 mg/m²: 25% reduction from 75 mg/m² |
| −3 (third dose reduction) | 45 mg/m²: 20% reduction from 56 mg/m² |
| −4 (fourth dose reduction) | 35 mg/m²: 25% reduction from 45 mg/m² |

If an AE resolves to Grade 1 or baseline at the reduced dose level, and no additional toxicities are observed during the following cycle of nab-sirolimus at the reduced dose, the dose may be increased to the previous dose level at the Investigator's discretion. nab-Sirolimus dose modification guidelines are outlined in Table 3 and Table 4 for clinically significant toxicities that are deemed related by the Investigator. The dosing schedule is described in the Schedule of Assessments (Table 2).

TABLE 4
Dose Modification Algorithms for Adverse Events Possibly Related to
nab-Sirolimus

| Adverse Reaction | Severity* | Dosage Modifications |
|---|---|---|
| Stomatitis | Grade 2 or 3 | Withhold nab-sirolimus until Grade ≤1. |
| | | Restart at the same dose for first occurrence. |
| | | If recurs, restart at reduced dose level. |
| | Grade 4 | Permanently discontinue nab-sirolimus. |
| Anemia | Grade 2 | Withhold nab-sirolimus until Hb ≥8 g/dL. |
| | | Restart at the same dose level. |
| | Grade 2:3 | Withhold nab-sirolimus until Hb ≥8 g/dL. |
| | | Restart at the same dose level. |
| | | If recurs, resume at reduced dose level. |
| Thrombocytopenia | Grade 2 | Withhold nab-sirolimus until platelet count |
| | | >100 × 10⁹/L. |
| | | Restart at the same dose level. |
| | Grade 2:3 | Withhold nab-sirolimus until platelet count |
| | | >100 × 10⁹/L. |
| | | Restart at reduced dose level. |
| Neutropenia | Grade 2 or 3 | Withhold nab-sirolimus until absolute |
| | | neutrophil count ≥1.5 × 10⁹/L. |
| | | Restart at the same dose level. |
| | Grade 4 | Withhold nab-sirolimus until absolute |
| | | neutrophil count ≥1.5 × 10⁹/L. |
| | | Restart at reduced dose level. |
| Infections | Grade 3 | Withhold nab-sirolimus until resolved. |
| | | Restart at reduced dose level. |
| | | If recurs, permanently discontinue nab- |
| | | sirolimus. |
| | Grade 4 | Withhold nab-sirolimus until resolved. |
| | | Restart at reduced dose level or permanently |
| | | discontinue nab-sirolimus. |
| Hypokalemia | Grade 2 | Withhold nab-sirolimus until Grade ≤1. |
| | | Restart at the same dose level. |
| | | If recurs, restart at reduced dose level. |
| | Grade ≥3 | Withhold nab-sirolimus until Grade ≤1. |
| | | Restart at reduced dose level. |
| | | If recurs, permanently discontinue nab- |
| | | sirolimus. |
| Blood bilirubin increased | Grade 2 | Withhold nab-sirolimus until Grade ≤1. |
| | | Restart at reduced dose level. |
| | | If recurs, permanently discontinue nab- |
| | | sirolimus. |
| | Grade ≥3 | Permanently discontinue nab-sirolimus. |
| Alanine aminotransferase | Grade 2 | Withhold nab-sirolimus until Grade ≤1. |
| (ALT) increased | | Restart at reduced dose level. |
| | | If recurs, permanently discontinue nab- |
| | | sirolimus. |
| | Grade ≥3 | Permanently discontinue nab-sirolimus. |
| Aspartate aminotransferase | Grade 2 | Withhold nab-sirolimus until Grade ≤1. |
| (AST) increased | | Restart at reduced dose level. |
| | | If recurs, permanently discontinue nab- |
| | | sirolimus. |
| | Grade ≥3 | Permanently discontinue nab-sirolimus. |
| Hyperglycemia | Grade ≥3 | Withhold nab-sirolimus until Grade ≤2. |
| | | Restart at reduced dose level. |
| Interstitial Lung Disease/ | Grade 2 | Withhold nab-sirolimus for up to 3 weeks |
| | | until Grade ≤1. |
| Non-Infectious | | Restart at reduced dose level. |
| Pneumonitis | | If not resolved to Grade ≤1 within 3 weeks, |
| | | permanently discontinue nab-sirolimus. |
| | | If recurs, permanently discontinue nab- |
| | | sirolimus. |
| | Grade 2:3 | Permanently discontinue nab-sirolimus. |
| Hemorrhage | Grade 2 or 3 | Withhold nab-sirolimus until Grade ≤1. |
| | | Resume at reduced dose. |
| | | If recurs, permanently discontinue nab- |
| | | sirolimus. |
| | Grade 4 | Permanently discontinue nab-sirolimus. |
| Other Adverse Reactions | Grade 3 | Withhold nab-sirolimus until Grade ≤1. |
| | | Restart at the same dose level. |
| | | If recurs, restart at reduced dose level. |
| | Grade 4 | Permanently discontinue nab-sirolimus. |

* Severity based on Common Terminology Criteria for Adverse Events Version 4.03.

Rechallenge may be considered if an alternative cause for impaired liver tests (ALT, AST, and alkaline phosphate) and/or elevated total bilirubin is discovered and the laboratory abnormalities resolve to normal or baseline. The decision to rechallenge the patient after nab-sirolimus has been held for potential hepatotoxicity is discussed and agreed upon unanimously by the patient, Investigator, and Aadi Medical Monitor. If signs or symptoms recur with rechallenge, nab-sirolimus is permanently discontinued. Patients who clearly meet the criteria for permanent discontinuation are never rechallenged.

On a per dose basis, an overdose is defined as 10% over the protocol-specified dose of nab-sirolimus assigned to a given patient, regardless of any associated AEs or sequelae. On a schedule or frequency basis, an overdose is defined as anything more frequent than the protocol required schedule or frequency. On an infusion rate basis, an overdose is defined as any rate faster than the protocol-specified rate of 30 minutes for each infusion.

nab-Sirolimus is reconstituted by designated study personnel per instructions noted in the Pharmacy Manual and administered to the patient at the study site. The Investigator or designee calculates the BSA of the patient per institutional standard methods in order to determine the total amount of nab-sirolimus to be administered. Dosing is capped at a BSA of 2 m². The calculated dosing volume of 5 mg/mL (rounded to the nearest mL) reconstituted nab-sirolimus suspension is administered by IV infusion over 30 minutes (+10 minutes window).

The letrozole used in this study is from commercial supply.

Patients receive full supportive care during the study, including transfusions of blood and blood products, and treatment with antibiotics, anti-emetics, anti-diarrheals, and analgesics, and other care as deemed appropriate, and in accordance with their institutional guidelines. White blood cell (WBC) and platelet growth factors may be administered at the discretion of the Investigator, consistent with institutional guidelines. Palliative radiation therapy or palliative surgery is not initiated during protocol participation except during the first 8 weeks of treatment, with Medical Monitor approval, and is allowed only in cases when other non-irradiated lesions are also followed for response beside the irradiated one(s). The need to initiate radiation therapy or palliative surgery beyond 8 weeks is considered non-protocol therapy and therefore a treatment failure. Extreme precaution must be taken with contraceptives (either combined or progesterone only), as it is not known if there is the potential of inhibition/induction of enzymes that affect the metabolism of estrogens and/or progestins.

The use of certain medications, and illicit drugs within 5 half-lives or 28 days, whichever is shorter prior to the first dose of IP and for the duration of the study is not allowed. The following medications or non-drug therapies are prohibited while receiving nab-sirolimus: other anticancer therapy while on treatment in this study, except as noted in the paragraph above; antiretroviral drugs (patients with known HIV are ineligible for study participation); herbal remedies (eg, St. John's wort) unless approval is granted by the Medical Monitor (tetrahydrocannabinol [THC] and cannabidiol [CBD]-containing products are permitted); use of strong inhibitors and inducers of CYP3A4 within 5 half-lives prior to receiving the first dose of nab-sirolimus. Sirolimus is metabolized primarily by CYP3A4 (drugs that are strong inhibitors or inducers of CYP3A4 may only be used under special circumstances (eg, as a single use for a procedure) while treatment with nab-sirolimus is interrupted; the list may be modified based on emerging data); use of any known CYP3A4 substrates with a narrow therapeutic window (such as fentanyl, alfentanil, astemizole, dihydroergotamine, pimozide, quinidine, or terfenadine) within 5 half-lives prior to receiving the first dose of nab-sirolimus unless discussed and agreed to by the Medical Monitor (single doses of fentanyl and similar agents are permissible if required for a procedure).

What is claimed is:

1. A method of treating an endometrioid endometrial cancer (EEC) in an individual in need thereof, the method comprising administering to the individual:
(a) a composition comprising nanoparticles comprising sirolimus and an albumin; and
(b) letrozole,
wherein letrozole is administered to the individual orally.

2. The method of claim 1, wherein letrozole is administered to the individual at an amount of about 0.1 mg to about 10 mg.

3. The method of claim 1, wherein letrozole is administered to the individual daily.

4. The method of claim 1, wherein the endometrioid endometrial cancer is locally advanced, advanced, malignant, advanced malignant, metastatic, or refractory, relapsed, recurrent, or resistant to a prior treatment.

5. The method of claim 4, wherein the prior treatment comprises (a) an mTOR inhibitor and/or a composition comprising nanoparticles comprising an mTOR inhibitor and an albumin, or (b) a platinum agent and/or a checkpoint inhibitor.

6. The method of claim 1, wherein the individual has not been treated with an mTOR inhibitor and/or a composition comprising nanoparticles comprising an mTOR inhibitor and an albumin.

7. The method of claim 1, wherein the EEC is FIGO Grade 1-2.

8. The method of claim 1, wherein sirolimus in the sirolimus nanoparticle composition is administered at an amount of about 10 mg/m² to about 150 mg/m².

9. The method of claim 8, wherein sirolimus in the sirolimus nanoparticle composition is administered at an amount of about 100 mg/m².

10. The method of claim 9, wherein the sirolimus nanoparticle composition is administered on days 1 and 8 of a 21-day cycle.

11. The method of claim 1, wherein the nanoparticle composition is characterized by one or more of: (a) the average diameter of the nanoparticles in the composition is no greater than about 150 nm, (b) the weight ratio of the albumin to sirolimus in the nanoparticle composition is no greater than about 9:1, or (c) the nanoparticles comprise sirolimus coated with the albumin.

12. The method of claim 1, wherein the sirolimus nanoparticle composition is administered intravenously.

13. The method of claim 1, wherein the sirolimus nanoparticle composition is administered concurrently with letrozole.

14. The method of claim 1, wherein the individual is chemotherapy-naïve.

15. The method of claim 1, wherein the EEC is unresectable.

16. The method of claim 1, wherein the individual has received no more than 1 line of chemotherapy.

17. The method of claim 1, wherein letrozole is administered at a dosage of about 2.5 mg.

18. The method of claim 9, wherein letrozole is administered daily at a dosage of about 2.5 mg.

19. The method of claim 10, wherein letrozole is administered daily at a dosage of about 2.5 mg.
